(19) United States
(12) Patent Application Publication
(10) Pub. No.: US 2003/0064989 A1
(43) Pub. Date: Apr. 3, 2003

(54) Inhibitors of cell proliferation, angiogenesis, fertility, and muscle contraction

(76) Inventors: Prudence Talbot, Redondo Beach, CA (US); Goar Melkonian, Riverside, CA (US); Karen Riveles, Riverside, CA (US); Lynn Ji, San Diego, CA (US)

Correspondence Address: KNOBBE MARTENS OLSON & BEAR LLP, 2040 MAIN STREET, FOURTEENTH FLOOR, IRVINE, CA, 92614, US

(21) Appl. No.: 10/153,450

(22) Filed: May 22, 2002

Related U.S. Application Data
(60) Provisional application No. 60/325,786, filed on Jun. 1, 2001.

(51) Int. Cl. A61K 31/53; A61K 31/501; A61K 31/498; A61K 31/519; A61K 31/506; A61K 31/444

(52) U.S. Cl. 514/242; 514/247; 514/252.01; 514/255.05; 514/256; 514/259.1; 514/336; 514/345; 514/352

(57) ABSTRACT

The invention concerns inhibitors of cell proliferation, angiogenesis, fertility, and muscle contraction, characterized by formula I

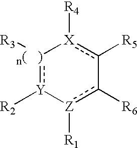

wherein,

X, Y and Z independently represent C or N;

------ is an optional double bond;

n is 0 or 1;

R₁, R₂, and R₄ independently represent hydrogen, a chemical bond, C₁₋₁₀ alkyl; C₂₋₁₀ alkenyl; C₂₋₁₀ alkinyl; aryl; aryl-C₁₋₁₀ alkyl; C₃₋₁₀ heterocyclyl; C₅₋₁₀ heteroaryl; halo, CF₃; NO₂; NHC(O)R*, OR, said alkyl, alkenyl, alkinyl, aryl, arylalkyl, heterocyclyl, or heteroaryl being optionally substituted;

R₃, R₅, and R₆ independently represent hydrogen, C₁₋₁₀alkyl; C₂₋₁₀ alkenyl; C₂₋₁₀ alkinyl; aryl; aryl-C₁₋₁₀alkyl; C₃₋₁₀ heterocyclyl; C₅₋₁₀ heteroaryl; halo, CF₃; NO₂; NHC(O)R*, OR, said alkyl, alkenyl, alkinyl, aryl, heterocyclyl, or heteroaryl being optionally substituted; or

R₅ and R₆ together form a 5- or 6-member aryl, heterocyclyl or heteroaryl group;

R is hydrogen or C₁₋₆ alkyl;

R* is hydrogen, or C₁₋₆ alkyl, or OH,

wherein the optional substituents are preferably selected from the group of one to three OH, C₁₋₆ alkyl, halo, NO₂, C₁₋₆ alkoxy, and CF3,

or a pharmaceutically acceptable salt thereof.

This application claims priority under Title 35, United States Codes §119(e) to provisional application No. 60/325,786 filed on Jun. 1, 2001, which is a conversion under 35 C.F.R. §111(b) and 37 C.F.R. §1.53(b)(2) of previous non-provisional application Ser. No. 09/872,602 filed on Jun. 1, 2001.

### BACKGROUND OF THE INVENTION

1. Field of the Invention

The present invention concerns inhibitors of cell proliferation, angiogenesis, fertility, and muscle contraction. In particular, the invention concerns the use of certain organic compounds identified in cigarette smoke, and their derivatives, in the treatment of diseases or pathological conditions associated with excessive, undesirable, or uncontrolled cell proliferation or angiogenesis, in fertility control, and as muscle relaxants.

2. Description of the Related Art

1. Effect and Components of Tobacco Smoke

There is solid scientific evidence that an association exists between the inhalation of tobacco smoke and a number of pathological conditions, including cardiopulmonary diseases and malignancies. The adverse effects of tobacco smoke on periodontal health, oviduct functioning, the abundance of vasculature in corpora lutea of females; ulcer healing and angiogenesis are also well documented. Villablanca et al., J. Appl. Physiol. 84:2089-1098 (1998); Magers et al., Reprod. Toxicol. 9:513-525 (1995); Dicarlantonio and Talbot, Biol Reprod. 61:651-656 (1999); Ma et al., Clin. Exp. Pharmacol. Physiol. 26:828-829 (1999); Knoll and Talbot, Reprod. Toxicol. 12:57-68 (1998).

More than 4,000 individual compounds have been identified in tobacco and tobacco smoke. Among these there are a number of compounds, such as tar, carbon monoxide, hydrogen cyanide, phenolic compounds, ammonia, formaldehyde, benzene, nitrosamines, nicotine, sulfur oxides, nitric oxides, benzo[a]pyrene, benzene compounds, etc., that have been identified as carcinogens or potential carcinogens. However, because of the complexity of smoke, identifying the compounds responsible for specific physiological effects is extremely difficult. Moreover, most studies involving cigarette smoke have been done using mainstream (MS) smoke, which is the bolus of smoke inhaled by active smokers, and paid little attention to sidestream (SS) smoke, the smoke produced at the burning end of a cigarette which is the main component in environmental tobacco smoke.

2. Role of Angiogenesis in Disease Pathogenesis

The formation of new blood vessels (angiogenesis) plays an important role in the pathogenesis of several diseases, including the development of solid tumors, and proliferative retinopathy. Tumors cannot grow and spread (metastasize) without the development of new blood vessels. Acquisition of a vascular supply enables tumors to procure oxygen and nutrients necessary for continued growth. Conversely, inhibition of angiogenesis has provided a promising new avenue for shrinking tumor growth and inhibiting metastasis.

In simple terms, the creation of new blood vessels occurs by a series of sequential steps. An endothelial cell forming the wall of an existing small blood vessel becomes activated, makes matrix metalloproteinase enzymes (MMPs) that break down the extracellular matrix, invades the matrix, and begins dividing. Eventually, strings of new endothelial cells organize into hollow tubes, creating new networks of blood vessels that make tissue growth and repair possible.

A number of proteins, including vascular endothelial growth factor (VEGF), acidic and basic fibroblast growth factors (aFGF and bFGF), angiogenin, epidermal growth factor (EGF), scatter factor (SF), placental growth factor (PIGF), interleukin-8 (IL-8), and tumor necrosis factor alpha (TNF-β), just to name a few, have been identified as activators of endothelial cell growth and/or movement. Other naturally occurring proteins, such as angiostatin, endostatin, interferons, platelet factor 4 (PF4), thrombospondin, transforming growth factor beta (TGF-β), 16Kd fragment of prolactin, and tissue inhibitor of metalloproteinase-1, -2, and -3 (TIMP-1, TIMP-2 and TIMP-3), are known as inhibitors of angiogenesis.

Clinical trials with various anti-angiogenic drugs, such as synthetic inhibitors of matrix metalloproteinases (MMP inhibitors), monoclonal antibodies targeting various growth factors involved in angiogenesis, such as VEGF, FGF, are at various stages of clinical trials. Other drugs in clinical trials, such as angiostatin, endostatin, squalamine, thalidomide, etc. inhibit endothelial cells directly.

For a review of anti-angiogenic chemotherapeutic agents see, for example, Schirner, Cancer Metastasis Rev. 19:67-73 (2000); Liekens et al., Biochem. Pharmacol. 61:253-70 (2001); and Ryan and Wilding, Drugs Aging 17:249-255 (2000).

### SUMMARY OF THE INVENTION

The present invention is based on the identification of several groups of compounds in cigarette smoke that inhibit cell proliferation, angiogenesis, oviductal functioning and/or muscle contraction in extremely low concentrations. While the compounds identified herein are harmful components of cigarette smoke, in appropriately low concentrations they, and their derivatives, find utility in the treatment of diseases or pathological conditions that require cell proliferation and/or angiogenesis, such as cancer and other cell proliferative diseases, in controlling fertility, and as muscle relaxants.

In one aspect, the invention concerns a method of inhibiting cell proliferation, comprising contacting the cell with a growth inhibitory amount of a compound of formula I

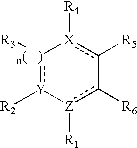

wherein

X, Y and Z independently represent C or N;

------ is an optional double bond;

n is 0 or 1;

R₁, R₂, and R₄ independently represent hydrogen, a chemical bond, C₁₋₁₀ alkyl; C₂₋₁₀ alkenyl; C₂₋₁₀ alkinyl; aryl; aryl-C₁₋₁₀ alkyl; C₃₋₁₀ heterocyclyl; C₅₋₁₀ heteroaryl; halo, CF₃; NO₂; NHC(O)R*, OR, said alkyl, alkenyl, alkinyl, aryl, arylalkyl, heterocyclyl, or heteroaryl being optionally substituted;

R₃, R₅, and R₆ independently represent hydrogen, C₁₋₁₀alkyl; C₂₋₁₀ alkenyl; C₂₋₁₀ alkinyl; aryl; aryl-C₁₋₁₀alkyl; C₃₋₁₀ heterocyclyl; C₅₋₁₀ heteroaryl; halo, CF₃; NO₂; NHC(O)R*, OR, said alkyl, alkenyl, alkinyl, aryl, heterocyclyl, or heteroaryl being optionally substituted; or

R₅ and R₆ together form a 5- or 6-member aryl, heterocyclyl or heteroaryl group;

R is hydrogen or C₁₋₆ alkyl;

R* is hydrogen, or C₁₋₆ alkyl, or OH,

wherein the optional substituents are preferably selected from the group of one to three OH, C₁₋₆ alkyl, halo, NO₂, C₁₋₆ alkoxy, and CF₃,

or a pharmaceutically acceptable salt thereof.

In formula I, the N heteroatom, if present, may optionally be oxidized or quaternized. Here and in all other definitions, when a variable (e.g. R₁, R₂, or R₄) is defined as a chemical bond, no substituent is attached to the affected position, rather a chemical bond is formed between the atom to which the variable is attached and an adjacent atom. Thus, for example, when Z is N, and R₁ is a chemical bond, no substituent is attached to Z, rather a double bond is formed between Z and an adjacent atom within the ring, such as the carbon to which R₆ is attached (i.e., the dotted line represents a double bond).

In another aspect, the invention concerns a method of inhibiting angiogenesis in a cell, comprising contacting the cell with an effective amount of a compound of formula I, wherein the variables are as defined above, or a pharmaceutically acceptable salt thereof.

In yet another aspect, the invention concerns a method of inhibiting the vascularization of endothelial cells, comprising contacting an endothelial cell, or a tissue or organ comprising endothelial cells, with an effective amount of a compound of formula I, wherein the variables are as defined above, or a pharmaceutically acceptable salt thereof.

In a further aspect, the invention concerns a method of treating a disease or condition associated with excessive, unwanted or uncontrolled cell proliferation and angiogenesis in a mammalian subject, comprising administering to the subject an effective amount of a compound of formula I, wherein the variables are as defined above, or a pharmaceutically acceptable salt thereof. Without limitation, the target disease or condition may, for example, be a tumor, such as cancer, a disease associated with corneal neovascularization, such as proliferative retinopathy, retinopathy of prematurity, corneal graft rejection, and neovascular glaucoma, arthritis, psoriasis, chronic inflammation, scleroderma, hemangioma, retrolental fibroplasia, abnormal capillary proliferation in hemophiliac joints, or prolonged menstruation and bleeding.

In a still further aspect, the invention concerns a method for prevention of contraception comprising administering to a female mammalian subject an effective amount of a compound of formula I, wherein the variables are as defined above, or a pharmaceutically acceptable salt thereof.

In a different aspect, the invention concerns a method for inducing abortion comprising administering to a female mammalian subject an effective amount of a compound of formula I, wherein the variables are as defined above, or a pharmaceutically acceptable salt thereof.

In a still further aspect, the invention concerns a method for controlling unwanted muscle contractions comprising administering to a mammalian subject an effective amount of a compound of formula I, wherein the variables are as defined above, or a pharmaceutically acceptable salt thereof.

In a further aspect, the invention concerns articles of manufacture for use in the methods defined above.

### BRIEF DESCRIPTION OF THE FIGURES

FIG. 1 shows that the C2 solid phase extraction cartridge retains most of the chick chorioallantoic membrane (CAM) growth inhibitory activity in sidestream gas phase smoke. CN shows growth (area) of the untreated control group. The SSG group is a positive control showing that SSG smoke inhibited CAM growth. The CC C2 group is a column control showing that eluent from an unused column does not affect CAM growth. SSG pt C2 contains the chemicals in SSG phase smoke that pass directly through the C2 cartridge. These chemicals did not significantly inhibit CAM growth. The eluent C2 group contains the chemicals that eluted off the C2 column after the SSG pass through has been collected. The eluent C2 group contains most of the inhibitory activity.

FIG. 2 is a gas chromatogram of sidestream whole smoke solution eluted from a solid phase extraction cartridge. A sidestream whole smoke solution was passed through the cartridge, which was then eluted with methanol, and the eluent was subjected to gas chromatography. Twelve pyridine derivatives were subsequently identified with mass spectrometry.

FIG. 3 shows representative video images of a control (A) and 2-ethylpyridine treated (B) CAM. Growth of the 2-ethylpyridine treated CAM has been significantly inhibited. Although not visible at this resolution, the capillary plexus has failed to form in the treated CAM.

FIG. 4A shows the activity of various concentrations of pyridine in the CAM cell proliferation assay.

FIG. 4B shows the activity of various concentrations of 4,4-bipyridine in the CAM cell proliferation assay.

FIG. 5A shows the activity of various concentrations of 2-methylpyridine in the CAM cell proliferation assay.

FIG. 5B shows the activity of various concentrations of 3-methylpyridine in the CAM cell proliferation assay.

FIG. 6A shows the activity of various concentrations of 2,3-dimethylpyridine in the CAM cell proliferation assay.

FIG. 6B shows the activity of various concentrations of 2,4-dimethylpyridine in the CAM cell proliferation assay.

FIG. 6C shows the activity of various concentrations of 2,4,6-trimethylpyridine in the CAM cell proliferation assay.

FIG. 7A shows the activity of various concentrations of 2-ethylpyridine in the CAM cell proliferation assay.

FIG. 7B shows the activity of various concentrations of 3-ethylpyridine in the CAM cell proliferation assay.

FIG. 7C shows the activity of lower concentrations of 3-ethylpyridine in the CAM cell proliferation assay.

FIG. 8 shows the activity of various concentrations of 4-ethenylpyridine in the CAM cell proliferation assay.

FIG. 9A shows the activity of various concentrations of β-nicotyrine in the CAM cell proliferation assay.

FIG. 9B shows the activity of various concentrations of nicotine in the CAM cell proliferation assay.

FIG. 9C shows the activity of various concentrations of nornicotine in the CAM cell proliferation assay.

FIGS. 10A-C shows that 2-ethylpyridine adversely affects angiogenesis in the chick chorioallantoic membrane (CAM). CAMs were exposed to 2-ethylpyridine on day 5 and evaluated on day 6:

(A) shows the effect of 2-ethylpyridine on the area of major blood vessels in the CAM;

(B) shows the effect of 2-ethylpyridine on the dendritic branching pattern;

(C) shows the effect of 2-ethylpyridine on the formation of the capillary plexus.

FIGS. 11A-C show the effect of 3-ethylpyridine on the oviduct:

(A) shows the effect of 3-ethylpyridine on oocyte pickup rate;

(B) shows the effect of 3-ethylpyridine on infundibular muscle contraction rate;

(C) shows the effect of 3-ethylpyridine on ciliary beat frequency.

### DETAILED DESCRIPTION OF THE PREFERRED EMBODIMENT

Definitions

Unless defined otherwise, technical and scientific terms used herein have the same meaning as commonly understood by one of ordinary skill in the art to which this invention belongs. For further information see, for example, Comprehensive Organic Chemistry, I. O. Sutherland editor, Pergamon Press, 1979; Vogel's Textbook of Practical Organic Chemistry, 5th Ed., 1989; Van Nostrand Reinhold, Encyclopedia of Chemistry, 4^{th} Ed., 1984; John McMurry, Organic Chemistry, 5^{th} Ed., 2000; Vollhardt and Schore, Organic Chemistry, W. H. Freeman and Co., New York, 1995. For purposes of the present invention, the following terms are defined below.

The term “mainstream smoke” or “MS smoke” is used to refer to cigarette smoke inhaled by active smokers. MS is generated during puff-drawing in the burning cone and hot zones of a cigarette, it travels through the tobacco column and is inhaled by the smoker. Although the smoke exhaled by the smoker is different from the smoke inhaled, it is also considered “mainstream” or “MS.”

The term “sidestream smoke” or “SS” is used to refer to the smoke formed between puff-drawing and emitted directly from the smoldering tobacco product into the air. This is the main component in environmental tobacco smoke.

The terms “environmental smoke,” “environmental tobacco smoke,” “ETS,” “secondhand smoke,” and “SHS” are used interchangeably, and refer to a mixture of the smoke emitted directly from the burning end of a smoldering tobacco product into the air (SS), and the smoke exhaled from the lungs of smokers.

“Angiogenesis” is defined as the promotion of the growth of new blood capillary vessels from existing endothelium. For further information, see also Folkman and Klagsbrun, “Angiogenic Factors” Science, 235:442-447 (1987); Folkman and Shing, “Angiogenesis” J. Biol. Chem., 267:10931-10934 (1992); J. Folkman, “Angiogenesis in Cancer, Vascular, Rheumatoid and Other Diseases,” Nature Medicine” 1: 27-31 (1995).

“Tumor”, as used herein, refers to all neoplastic cell growth and proliferation, whether malignant or benign, and all pre-cancerous and cancerous cells and tissues.

The terms “cancer” and “cancerous” refer to or describe the physiological condition in mammals that is typically characterized by unregulated cell growth. Examples of cancer include but are not limited to, carcinoma, lymphoma, blastoma, sarcoma, and leukemia. More particular examples of such cancers include breast cancer, prostate cancer, colon cancer, squamous cell cancer, small-cell lung cancer, non-small cell lung cancer, gastrointestinal cancer, pancreatic cancer, cervical cancer, ovarian cancer, liver cancer, e.g. hepatic carcinoma, bladder cancer, colorectal cancer, endometrial carcinoma, kidney cancer, and thyroid cancer.

The term “metastasis” refers to the process by which tumor cells are spread to distant parts of the body. The term is also used herein to refer to a tumor that develops through the metastatic process.

“Proliferative retinopathy” is a rapid and abnormal growth of new small blood vessels of the retina. These blood vessels are fragile and liable to bleed, with subsequent deterioration of vision, and may lead to blindness through haemorrhage and scarring. It is usually the complication of diabetes.

A “disorder” is any condition that would benefit from treatment of the present invention. This includes chronic and acute disorders or diseases including those pathological conditions which predispose the mammal to the disorder in question.

The terms “treat” or “treatment” refer to both therapeutic treatment and prophylactic or preventative measures, wherein the object is to prevent or slow down (lessen) an undesired physiological change or disorder, such as the development or spread of cancer. For purposes of this invention, beneficial or desired clinical results include, but are not limited to, alleviation of symptoms, diminishment of extent of disease, stabilized (i.e., not worsening) state of disease, delay or slowing of disease progression, amelioration or palliation of the disease state, and remission (whether partial or total), whether detectable or undetectable. “Treatment” can also mean prolonging survival as compared to expected survival if not receiving treatment. Those in need of treatment include those already with the condition or disorder as well as those prone to have the condition or disorder or those in which the condition or disorder is to be prevented.

The term “(therapeutically) effective amount” refers to an amount of a drug effective to treat a disease or disorder in a mammal. In the case of cancer, the therapeutically effective amount of the drug may reduce the number of cancer cells; reduce the tumor size; inhibit (i.e., slow to some extent and preferably stop) cancer cell infiltration into peripheral organs; inhibit (i.e., slow to some extent and preferably stop) tumor metastasis; inhibit, to some extent, tumor growth; and/or relieve, to some extent, one or more of the symptoms associated with the cancer. To the extent the drug may prevent growth and/or kill existing cancer cells, it may be cytostatic and/or cytotoxic. For cancer therapy, efficacy can, for example, be measured by assessing the time to disease progression (TTP) and/or determining the response rate (RR).

The term “growth inhibitory amount” when used herein refers to an amount which inhibits growth of a target cell, such as a tumor cell, either in vitro or in vivo, irrespective of the mechanism by which cell growth is inhibited. In a preferred embodiment, the growth inhibitory amount inhibits growth of the target cell in cell culture by greater than about 20%, preferably greater than about 50%, most preferably greater than about 75% (e.g. from about 75% to about 100%).

The term “cytotoxic agent” as used herein refers to a substance that inhibits or prevents the function of cells and/or causes destruction of cells. The term is intended to include radioactive isotopes (e.g. At²¹¹, I¹³¹, I¹²⁵, Y⁹⁰, Re¹⁸⁶, Re¹⁸⁸, Sm¹⁵³, Bi²¹², P³² and radioactive isotopes of Lu), chemotherapeutic agents, toxins such as small molecule toxins or enzymatically active toxins of bacterial, fungal, plant or animal origin, and antibodies, including fragments and/or variants thereof.

A “chemotherapeutic agent” is a chemical compound useful in the treatment of cancer, such as, for example, taxanes, e.g. paclitaxel (TAXOL®, Bristol-Myers Squibb Oncology, Princeton, N.J.) and doxetaxel (TAXOTERE®, Rh6ne-Poulenc Rorer, Antony, France), chlorambucil, vincristine, vinblastine, anti-estrogens including for example tamoxifen, raloxifene, aromatase inhibiting 4(5)-imidazoles, 4-hydroxytamoxifen, trioxifene, keoxifene, LY117018, onapristone, and toremifene (Fareston), and anti-androgens such as flutamide, nilutamide, bicalutamide, leuprolide, and goserelin, etc.

The term “alkyl” refers to a monovalent alkane (hydrocarbon) derived radical containing from 1 to 10 carbon atoms unless otherwise defined. It may be straight- or branched-chained, or cyclic. Preferred straight- or branched-chained alkyl groups include methyl, ethyl, propyl, isopropyl, butyl, and t-butyl. Preferred cycloalkyl groups include cyclopropyl, cyclobutyl, cycloheptyl, cyclopentyl, and cyclohexyl. The term “lower alkyl” refers to alkyl groups as hereinabove defined, having 1 to 6 carbon atoms. The term “alkyl” as used herein includes substituted alkyls.

The term “substituted alkyl” refers to alkyl as defined above, including one or more functional groups such as lower alkyl, aryl, acyl, halogen, hydroxy, amino, alkoxy, alkylamine, acylamino, acyloxy, aryloxy, aryloxyalkyl, mercapto, both saturated and unsaturated cyclic hydrocarbons, heterocycles, and the like. These groups may be attached to any carbon of the alkyl moiety.

The term “aryl” is used herein to refer to an aromatic substituent which may be a single aromatic ring or multiple aromatic rings which are fused together, linked covalently, or linked to a common group such as a methylene or ethylene moiety. The common linking group may also be a carbonyl as in benzophenone. The aromatic ring(s) may include phenyl, naphthyl, biphenyl, diphenylmethyl and benzophenone among others. The term “aryl” encompasses “arylalkyl.” The term “aryl” as used herein also includes substituted aryl.

“Substituted aryl” refers to aryl, as defined above, including one or more functional groups such as lower alkyl, acyl, halogen, alkylhalos, hydroxy, amino, alkoxy, alkylamine, acylamino, acyloxy, mercapto and both saturated and unsaturated cyclic hydrocarbons which are fused to the aromatic ring(s), linked covalently or linked to a common group such as a methylene or ethylene moiety. The linking group may also be a carbonyl such as in cyclohexyl phenyl ketone. The term “substituted aryl” encompasses “substituted arylalkyl.”

The term “heterocycle,” “heteroaryl,” or “heterocyclic,” as used herein, unless noted otherwise, represents a stable 5- to 7-member mono- or 7- to 10-member bicyclic heterocyclic ring system, any ring of which may be saturated or unsaturated, aromatic or non-aromatic, and which consists of carbon atoms and from one to three heteroatoms selected from N, O and S. The N and S atoms may be optionally oxidized, and the N heteroatom may be optionally quaternized. Heterocycles include any bicyclic group in which any of the above groups is fused to a benzene ring. Examples of heterocyclic groups (inlcuding heteroaryls) are, without limitation, pyridyl, pyrazinyl, pyrimidinyl, pyridazinyl, oxazolyl, oxazolidinyl, isoxazolyl, isoxazolidinyl, morpholinyl, thiazolyl, thiazolidinyl, isothiazolyl, quinuclidinyl, isothiazolidinyl, indolyl, quinolinyl, isoquinolinyl, benzimidazolyl, thiadiazoyl, benzopyranyl, benzothiazolyl, benzoxazolyl, furyl, tetrahydrofuryl, tetrahydropyranyl, thiophenyl, imidazopyridinyl, tetrazolyl, triazinyl, thienyl, benzothienyl, thiamorpholinyl sulfoxide, thiamorpholinyl sulfone, oxadiazolyl, include piperidinyl, piperazinyl, 2-oxopiperazinyl, 2-oxopiperidinyl, 2-oxopyrrolodinyl, 2-oxoazepinyl, azepinyl, pyrrolyl, 4-piperidonyl, pyrrolidinyl, pyrazolyl, pyrazolidinyl, imidazolyl, imidazolinyl, and imidazolidinyl.

The term “aralkyl” or “arylalkyl” is used to refer to an aryl or heteroaryl moiety, as defined herein, attached through a C₁₋₆ alkyl linker, where alkyl is as defined above.

The term “alkoxy” refers to a substituent with a straight- or branched-chain alkyl, alkenyl, or alkinyl group of the designated length, which is attached via an oxygen molecule. Representative alkoxy groups are methoxy, ethoxy, propoxy, isopropoxy, butoxy, isobutoxy, t-butoxy, pentoxy, isopentoxy, hexoxy, isohexoxy, allyloxy, propargyloxy, vinyloxy, etc.

The term “halogen” is used herein to refer to fluorine, bromine, chlorine and iodine atoms.

The term “amino” is used to refer to the group —NRR′, where R and R′ may independently be hydrogen, alkyl, substituted alkyl, aryl, substituted aryl or acyl.

The term “alkoxy” is used herein to refer to an —OR group, where R is a lower alkyl, substituted lower alkyl, aryl, substituted aryl, arylalkyl or substituted arylalkyl wherein the alkyl, aryl, substituted aryl, arylalkyl and substituted arylalkyl groups are as described herein. Suitable alkoxy radicals include, for example, methoxy, ethoxy, phenoxy, substituted phenoxy, benzyloxy, phenethyloxy, t-butoxy, etc.

The term “alkenyl” is used herein to refer to an unsaturated straight- or branched-chained, or cyclic monovalent hydrocarbon radical having at least one carbon-carbon double bond. The radical can be in either the cis or trans conformation about the double bond(s). Suitable alkenyl radicals include, for example, ethenyl, propenyl, isopropenyl, cyclopropenyl, butenyl, isobutenyl, cyclobutenyl, tert-butenyl, pentenyl, hexenyl, etc.

The term “alkynyl” is used herein to refer to an unsaturated branched, straight chain or cyclic monovalent hydrocarbon radical having at least one carbon-carbon triple bond. Suitable alkynyl radicals include, for example, ethynyl, propynyl, butynyl, isobutynyl, pentynyl, hexynyl, etc.

The term “pharmaceutically acceptable salt” refers to those salts of compounds which retain the biological effectiveness and properties of the free bases and which are obtained by reaction with inorganic acids such as hydrochloric acid, hydrobromic acid, sulfuric acid, nitric acid, phosphoric acid, methanesulfonic acid, ethanesulfonic acid, p-toluenesulfonic acid, salicylic acid and the like. Pharmaceutically acceptable salts include, for example, alkali metal salts, such as sodium and potassium, alkaline earth metal salts and ammonium salts.

The term “prodrug,” as used herein, represents compounds which are rapidly transformed in vivo to the parent compounds of the present invention, or to their derivatives, for example, by hydrolysis in blood. A thorough discussion is provided in Higuchi and Stella, Pro-drugs as Novel Delivery Systems, Vol. 14, A.C.S. Symposium Series, and in Bioreversible Carriers in Drug Design, Edward B. Roche, ed., American Pharmaceutical Association and Pergamon Press, 1987.

The Compounds

The present invention is based on the identification of certain very potent inhibitors of cell proliferation, angiogenesis, and oviduct functioning in tobacco smoke. In particular, the present invention concerns the beneficial use of certain heterocyclic compounds, which are potent inhibitors of cell proliferation, angiogenesis and/or oviduct functioning, and/or act as muscle relaxants.

The compounds of the present invention are described by formula I

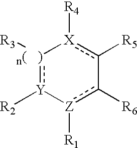

wherein

X, Y and Z independently represent C or N;

------ is an optional double bond;

n is 0 or 1;

R₁, R₂, and R₄ independently represent hydrogen, a chemical bond, C₁₋₁₀ alkyl; C₂₋₁₀ alkenyl; C₂₋₁₀ alkinyl; aryl; aryl-C₁₋₁₀ alkyl; C₃₋₁₀ heterocyclyl; C₅₋₁₀ heteroaryl; halo, CF₃; NO₂; NHC(O)R*, OR, said alkyl, alkenyl, alkinyl, aryl, arylalkyl, heterocyclyl, or heteroaryl being optionally substituted;

R₃, R₅, and R₆ independently represent hydrogen, C₁₋₁₀alkyl; C₂₋₁₀ alkenyl; C₂₋₁₀ alkinyl; aryl; aryl-C₁₋₁₀alkyl; C₃₋₁₀ heterocyclyl; C₅₋₁₀ heteroaryl; halo, CF₃; NO₂; NHC(O)R*, OR, said alkyl, alkenyl, alkinyl, aryl, heterocyclyl, or heteroaryl being optionally substituted; or

R₅ and R₆ together form a 5- or 6-member aryl, heterocyclyl or heteroaryl group;

R is hydrogen or C₁₋₆ alkyl;

R* is hydrogen, or C₁₋₆ alkyl, or OH,

wherein the optional substituents are preferably selected from the group of one to three OH, C₁₋₆ alkyl, halo, NO₂, C₁₋₆ alkoxy, and CF₃,

or a pharmaceutically acceptable salt thereof.

In formula I, the N heteroatom may optionally be oxidized or quaternized.

A preferred subgenus of the compounds of the present invention is characterized by the formula Ia,

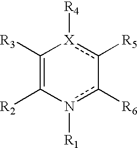

wherein

X is C or N;

------ is an optional double bond;

R₁ and R₄ independently represent hydrogen, a chemical bond, C₁₋₁₀alkyl; C₂₋₁₀ alkenyl; C₂₋₁₀ alkinyl; aryl; aryl-C₁₋₁₀alkyl; C₃₋₁₀ heterocyclyl; C₅₋₁₀ heteroaryl; halo, CF₃; NO₂; NHC(O)R*, OR, said alkyl, alkenyl, alkinyl, aryl, arylalkyl, heterocyclyl, or heteroaryl being optionally substituted;

R₂, R₃, R₅, and R₆ independently represent hydrogen, C₁₋₁₀alkyl; C₂₋₁₀ alkenyl; C₂₋₁₀ alkinyl; aryl; aryl-C₁₋₁₀alkyl; C₃₋₁₀ heterocyclyl; C₅₋₁₀ heteroaryl; halo, CF₃; NO₂; NHC(O)R*, OR, said alkyl, alkenyl, alkinyl, aryl, heterocyclyl, or heteroaryl being optionally substituted; or

R₅ and R₆ together form a 5- or 6-member aryl, heterocyclyl or heteroaryl group;

R is hydrogen or C₁₋₆ alkyl;

R* is hydrogen, or C₁₋₆ alkyl, or OH,

wherein the optional substituents are preferably selected from the group of one to three OH, C₁₋₆ alkyl, halo, NO₂, C₁₋₆ alkoxy, and CF₃.

In a preferred group of the compounds of formula Ia herein Z is a carbon atom, at least one of R₂, R₃, and R₄ is optionally substituted C₁₋₆ alkyl, optionally substituted C₂₋₁₀ alkenyl, or optionally substitued C₂₋₁₀ alkinyl, while the other(s) is/are hydrogen, OH, or C₁₋₆ alkoxy; and R₅ and R₆ are both hydrogen, or together form a benzene ring to yield a quinoline structure.

In a particularly preferred group of the compounds of formula Ia herein Z is a carbon atom, one, two or three of R₂, R₃, and R₄ is C₁₋₆ alkyl, C₂₋₁₀ alkenyl, or C₂₋₁₀ alkinyl, while the other(s) is/are hydrogen; and R₅ and R₆ are both hydrogen.

In another preferred group of the compounds of formula Ia herein Z is a carbon atom, R₁ is hydrogen, one of R₂, R₃, R₅, and R₆ is a heteroaryl or heterocyclyl group, wherein the heteroatom preferably is nitrogen, and the heteroaryl group preferably is six membered, while the heterocyclyl group preferably is five membered.

A further preferred subgenus of the compounds herein is represented by the formula Ib

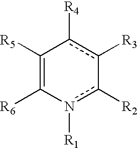

wherein R₁, R₂, R₃, R₄, R₅, and R₆ are as defined above, in connection with formula Ia.

Another preferred subgenus of the compounds herein is represented by the formula Ic

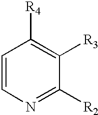

wherein R₂, R₃, and R₄ are as defined above, in connection with formula Ia.

Examples of the compounds of the invention, without limitation, are:

2-methylpyridine,

3-methylpyridine,

4-methylpyridine,

2,3-dimethylpyridine,

2,4-dimethylpyridine,

3,4-dimethylpyridine,

2,3,4-trimethylpyridine,

2,3,5-trimethylpyridine,

2,3,6-trimethylpyridine,

2,4,5-trimethylpyridine,

2,4,6-trimethylpyridine,

3,4,5-trimethylpyridine,

3,4,6-trimethylpyridine,

2-ethylpyridine,

3-ethylpyridine,

4-ethylpyridine,

2,3-diethylpyridine,

2,4-diethylpyridine,

3,4-diethylpyridine,

2,3,4-triethylpyridine,

2,3,5-triethylpyridine,

2,3,6-triethylpyridine,

2,4,5-triethylpyridine,

2,4,6-triethylpyridine,

3,4,5-triethylpyridine,

3,4,6-triethylpyridine,

2-methyl-3-ethyl-pyridine,

2-metyl-4-ethyl-pyridine,

3-methyl-4-ethyl-pyridine,

2-methyl-3,4 diethyl-pyridine,

2-methyl-3,5-diethyl-pyridine,

2-methyl-3,6-diethyl-pyridine,

2-methyl-4,5-diethyl-pyridine,

2-methyl-4,6-diethyl-pyridine,

3-methyl-4,5-diethyl-pyridine,

3-methyl-4,6-diethyl-pyridine,

2-ethyl-3-methyl-pyridine,

2-ethyl-4-methyl-pyridine,

3-ethyl-4-methyl-pyridine,

2-ethyl-3,4-dimethyl-pyridine,

2-ethyl-3,5-dimethyl-pyridine,

2-ethyl-3,6-dimethyl-pyridine,

2-ethyl-4,5-dimethyl-pyridine,

2-ethyl-4,6-dimethyl-pyridine,

3-ethyl-4,5-dimethyl-pyridine,

3-ethyl-4,6-dimethyl-pyridine,

3-ethenylpyridine,

4-ethenylpyridine,

4,4-bipyridine,

3-1-methylpyrrol-2-yl) pyridine (nicotyrine),

1-methyl-2-(3-pyridyl)pyrrolidine (nicotine),

3-(1-pyrrilidin-2-yl)pyridine (nornicotine),

2-methylpyrazine,

3-methylpyrazine,

2,3-dimethylpyrazine,

2,5-dimethylpyrazine,

2,6-dimethylpyrazine,

2,3,5-trimethylpyrazine,

2,3,6-trimethylpyrazine,

2-ethylpyrazine,

3-ethylpyrazine,

2,3-diethylpyrazine,

2,5-diethylpyrazine,

2,6-diethylpyrazine,

2,3,5-triethylpyrazine,

2,3,6-triethylpyrazine,

2-methyl-3-ethyl-pyrazine,

2-ethyl-3-methyl-pyrazine,

2-methyl-5-ethyl-pyrazine,

2-ethyl-5-methyl-pyrazine,

2-methyl-6-ethyl-pyrazine,

2-ethyl-6-methyl-pyrazine,

2,3-dimethyl-5-ethyl-pyrazine,

2,3-dimethyl-6-ethyl-pyrazine,

2,3-diethyl-5-methyl-pyrazine,

2,3-diethyl-6-methyl-pyrazine,

2-ethenylpyrazine,

3-ethenylpyrazine;

1-methyl-pyrazole;

1-ethyl-pyrazole;

1,4-dimethyl-pyarzole;

1,4-diethyl-pyrazole;

trimethylpyrazole;

triethylpyrazole;

phenol,

2-methyl-phenol;

2-ethyl-phenol;

3-methyl-phenol;

3-ethyl-phenol;

4-methyl-phenol;

4-ethyl-phenol;

quinoline;

isoquinoline;

indole;

cyclopentanone;

cyclopenten-1-one;

2-methyl-cyclopenten-1-one;

1-piperazine-ethanol.

In addition, the invention includes the use of cyclopentanones, benzene and several alcohol derivatives found in various components of cigarette smoke, such as cyclopentanone, 2-cyclopenten-1-one, 2-methyl-ethanone, 1-(2-furanyl)phenylethyl alcohol, etc. in the treatment methods and compositions herein.

Although the compounds used in accordance with the present invention were originally isolated from tobacco smoke, they are commercially available or can be readily synthesized by well known reactions of organic chemistry, following methods known by those skilled in the art and described in standard textbooks of organic chemistry, such as, for example, Comprehensive Organic Chemistry, I. O. Sutherland editor, Pergamon Press, 1979.

For example, pyridines and simple alkylpyridines are typically obtained from coal tar, while more highly substituted pyridines are usually made by both electrophilic and nucleophilic substitution of the simpler derivatives. Thus pyridines can be made by condensation reactions of acyclic starting materials, such as carbonyl compounds with ammonia, for example by the Hantsch pyridine synthesis. In this reaction, two molecules of a β-dicarbonyl compound, an aldehyde, and ammonia combine in several steps to give a substituted dihydropyridine, which is readily oxidized by nitric acid to the aromatic system. Further derivatives may be made, for example, by nucleophilic substitution, which is a preferred way of synthesizing aminopyridine, phenylpyridine, alkoxypyridine derivatives. Alternative reaction schemes, conditions, and protocols would be apparent to those skilled in the art.

In accordance with standard methods well known to those skilled in the art, the compounds specifically disclosed herein can be used as lead compounds to create analog compounds. Analog compounds possess similar biological functions, but differ in their chemical structure. Such compounds may be created by substituting different functional groups, such as alkyl, alkenyl, acetyl, amino or hydroxyl groups, for those already present on the parent compounds. Additional functional groups may also be added to the parent compounds. The resulting compounds may exhibit more desirable therapeutic properties, or be more pharmacologically active, than the parent molecules.

Standard textbooks, such as “Principles of Medicinal Chemistry,” William O. Foye, Thomas L. Lemke, and David A. Williams, editors, 4th ed., 1995, may be consulted to produce such compounds, without undue experimentation.

The present invention includes the use of pharmaceutically acceptable salts of the pyridine derivatives disclosed. Preferred pharmaceutically acceptable salts are those which favorably affect the physical and/or pharmacokinetic properties of the compound, such as solubility, absorption, distribution, bioavailability, metabolism, and excretion. Pharmaceutically acceptable salts includes salts with alkali metal cations, such as sodium and potassium salts, and with other suitable counterions, e.g. tetramethylammonium, tetrabutylammonium, choline, triethylammonium, triethanol-hydroammonium, etc. Pharmaceutically acceptable salts also include acid addition salts derived from inorganic or organic acids, such as acetate, adipate, alginate, aspartate, benzoate, benzenesulfonate, bisulfate, butyrate, citrate, camphorate, camphorsulfonate, cyclopentanepropionate, digluconate, dodecylsulfate, ethanesulfonate, fumarate, glucoheptanoate, glycerophosphate, hemisulfate, heptanoate, hexanoate, hydrochloride, hydrobromide, hydroiodide, 2-hydroxy-ethanesulfonate, lactate, maleate, methanesulfonate, 2-naphthalenesulfonate, nicotinate, oxalate, pamoate, pectinate, persulfate, 3-phenylpropionate, picrate, pivalate, propionate, succinate, tartrate, thiocyanate, tosylate and undecanoate. Base salts include ammonium salts, alkali metal salts such as sodium and potassium salts, alkaline earth metal salts such as calcium and magnesium salts, salts with organic bases such as dicyclohexylamine salts, N-methyl-D-glucamine, and salts with amino acids such as arginine, lysine, and so forth.

The compounds herein may have asymmetric centers and occur as racemates, racemic mixtures and as individual diastereomers, or enantiomers with all isomeric forms being included in the present invention. When any variable, e.g. alkyl, alkenyl, aryl, etc. group occurs more than one time in any constituent or in Formula I, its definition on each occurrence is independent of its definition at every other occurrence, unless otherwise stated.

Uses of the Compounds

The compounds of the present invention find utility in the inhibition of cell proliferation and angiogenesis, in fertility control, and in controlling muscle contractions.

Cell proliferation and/or angiogenesis play an important role in the pathogenesis of numerous diseases, including cancer. The methods of this invention are effective in inhibiting malignant tumor growth, and angiogenesis associated with malignant tumor growth. This includes cancerous tumor growth on cells tissues and organs. Tumors, the growth and/or spread of which can be controlled in accordance with the present invention, include, without limitation, breast cancer, prostate cancer, colon cancer, squamous cell cancer, small-cell lung cancer, non-small cell lung cancer, gastrointestinal cancer, pancreatic cancer, cervical cancer, ovarian cancer, liver cancer, bladder cancer, colorectal cancer, endometrial carcinoma, kidney cancer, thyroid cancer, hepatic carcinoma, and the like.

In addition to the pathology of the growth and spread of solid tumors, angiogenesis and/or abnormal cell proliferation are involved in a variety of other diseases and conditions, such as, for example, diseases associated with corneal neovascularization, including proliferative (e.g. diabetic) retinopathy, which has been discussed before, retinopathy of prematurity, corneal graft rejection, and neovascular glaucoma. Protracted angiogenesis is observed also in arthritis, psoriasis, chronic inflammation, scleroderma, hemangioma, retrolental fibroplasia and abnormal capillary proliferation in hemophiliac joints, prolonged menstruation and bleeding, and other disorders of the female reproductive system. In many of these diseases, unrestrained new capillary growth itself contributes to the disease process.

For example, in arthritis, new capillaries may invade and destroy joint cartilage, and inhibition of this angiogenesis may help in controlling the disease.

Psoriasis is characterized by a hyperproliferation of the basal cells (a several fold increase in the number of basal cells of the epidermis). This increase in the basal cell population reduces the turnover time of the epidermis from the normal 27 days to 3-4 days. This shortened interval prevents normal cell maturation and keratinization, and this failure of maturation is reflected in an array of abnormal morphologic and biochemical changes. Accordingly, control of cell proliferation offers promises in the treatment of psoriasis. In addition, the prevention of the formation of new blood vessels necessary to maintain the characteristic psoriatic lesions, leads to relief from symptoms.

In view of the knowledge that oviductal functioning and angiogenesis in corpora lutea are affected by inhalation of or in vitro exposure to mainstream and sidestream cigarette smoke, and the data disclosed herein, the compounds of the present invention are also believed to find utility in fertility control, including prevention of conception, interruption of pregnancy, and induction of abortion, preferably early in pregnancy, regardless of the mechanism involved. Specifically included are contraceptives the effect of which is based on the inhibition of implantation (receptivity inhibition).

There are various conditions that require or benefit from the control of (smooth) muscle contractions, such as, for example, premature uterine contractions during pregnancy, unwanted bladder or gut contractions. For example, in premature labor the uterine musculature is triggered by various stimuli to contract at a time when it is undesirable or even life-threatening to do so. The compounds of the present invention are believed to be useful in preventing uterine contractions in the case of premature labor, or other conditions when delay of labor might be desirable. Another condition, treatable with muscle relaxants is urinary incontinence, which affects approximately 13 million people in the United States, most of them older adults and twice as many women as men. Epidemiologic studies estimate that 15-35% of non institutionalized persons over age 60 have incontinence. This figure rises to 50% in the nursing home population. Although the prevalence of urinary incontinence increases with age, it is not considered a normal part of aging. The compounds of the present invention, due to their muscle relaxant properties, are believed to find use in the treatment of urinary incontinence.

Pharmaceutical Compositions

The pharmaceutical compositions of the present invention comprise a compound of formula I, or a pharmaceutically acceptable salt thereof, as active ingredient, in admixture with a pharmaceutically acceptable carrier and optionally other ingredients.

Pharmaceutical compositions for use in accordance with the present invention can be formulated in conventional manner using one or more physiologically acceptable carriers or excipients. Thus, the compounds and their pharmaceutically acceptable salts and solvates can be formulated for administration orally, topically, parenterally, by inhalation or insufflation (either through the mouth or the nose), buccally, or rectally.

For oral administration, the pharmaceutical compositions can take the form of, for example, tablets, capsules, including soft and hard gelatine capsules, troches, lozenges, aqueous or oily suspensions, dispersible powders or granules, emulsions, etc. prepared by conventional means with pharmaceutically acceptable excipients such as solvents, suspending agents, diluents, binding agents (e.g., pregelatinised maize starch, polyvinylpyrrolidone or hydroxypropyl methylcellulose); fillers (e.g., lactose, microcrystalline cellulose or calcium hydrogen phosphate); lubricants (e.g., magnesium stearate. talc or silica); disintegrants (e.g., potato starch or sodium starch glycolate); or wetting agents (e.g., sodium lauryl sulfate). The tablets can be coated by methods well known in the art. Liquid preparations for oral administration can take the form of, for example, solutions, syrups or suspensions, or they can be presented as a dry product for constitution with water or other suitable vehicle before use. Such liquid preparations can be prepared by conventional means with pharmaceutically acceptable additives such as suspending agents (e.g., sorbitol syrup, cellulose derivatives or hydrogenated edible fats); emulsifying agents (e.g., lecithin or acacia); non-aqueous vehicles (e.g., almond oil, oily esters, ethyl alcohol or fractionated vegetable oils); and preservatives (e.g., methyl or propyl-p-hydroxybenzoates or sorbic acid). The preparations can also contain buffer salts, flavoring, coloring and sweetening agents as appropriate.

Preparations for oral administration can be suitably formulated to give controlled release of the active compound. For buccal administration the compositions can take the form of tablets or lozenges formulated in conventional manner.

For topical administration, creams, ointments, gels, solutions, or suspensions, etc., containing a compound of formula I can be used. Topical formulations typically include pharmaceutically acceptable carriers, solvents, emulsifiers, penetration enhancers, preservatives, emollients, and the like.

For administration by inhalation, the compounds for use according to the present invention are conveniently delivered in the form of an aerosol spray presentation from pressurized packs or a nebulizer, with the use of a suitable propellant, e.g., dichlorodifluoromethane, trichlorofluoromethane, dichlorotetrafluoroethane, carbon dioxide or other suitable gas. In the case of a pressurized aerosol the dosage unit can be determined by providing a valve to deliver a metered amount. Capsules and cartridges of e.g., gelatin for use in an inhaler or insufflator can be formulated containing a powder mix of the compound and a suitable powder base such as lactose or starch.

The compounds can be formulated for parenteral administration. The term parental is used to include subcutaneous, intravenous, intramuscular, intrasternal injection or infusion techniques, including bolus injection and continuous infusion. Formulations for injection can be presented in unit dosage form, e.g., in ampoules or in multi-dose containers, with an added preservative. The compositions can take such forms as suspensions, solutions or emulsions in oily or aqueous vehicles, and can contain formulatory agents such as suspending, stabilizing or dispersing agents. Alternatively, the active ingredient can be in powder form for constitution with a suitable vehicle, e.g., sterile pyrogen-free water, before use. The compounds can also be formulated in rectal compositions such as suppositories or retention enemas, e.g., containing conventional suppository bases such as cocoa butter or other glycerides.

The compounds herein have been found to be effective in very small amounts, a typical effective concentration in a liquid formulation will typically be in the range of 10⁻⁶-10⁻¹⁵ moles per liter, more preferably 10⁻⁹-10⁻¹⁵ moles per liter, most preferably 10⁻¹²-10⁻¹⁵ moles per liter.

In order to obtain consistency of administration, it is preferred that a composition of the invention is in the form of a unit dose. Suitable unit dose forms include ampoules, tablets, capsules and powders in sachets or vials. Such unit dose forms may contain, for example, from 0.001 to 1000 mg of a compound of the invention and preferably from 0.1 to 50 mg. Still further preferred unit dosage forms contain 1 to 25 mg of a compound of the present invention. The compounds of the present invention can be administered orally at a dose range of about 0.001 to 100 mg/kg, preferably at a dose range of 0.01 to 10 mg/kg, more preferably at a dose range of 0.1 to 5 mg/kg, however, doses outside these ranges are also possible. The determination of the effective dose is well within the skill of an ordinary physician, and depends on factors like the nature of the disease or condition to be treated, the stage or progression of the disease, the patient's general health condition, age, sex, etc.

Treatment Methods

Drugs that can inhibit cell proliferation and angiogenesis are very valuable in controlling diseases accompanied by excessive cell proliferation and/or angiogenesis, such as cancer, psoriasis, proliferative retinopathy, and other diseases discussed above in great detail. The compounds disclosed herein are particularly valuable in treating such diseases, as they are very strong inhibitors of both cell proliferation and angiogenesis at extremely low (femtomolar to nanomolar) concentrations. Most anti-cancer drugs target one process participating in the development, growth and/or spread of cancer, such as angiogenesis. By effectively shutting down both cell division and angiogenesis, the compounds disclosed herein hold great promise in the treatment of diseases the pathogenesis of which involves both cell division and angiogenesis, such as cancer, psoriasis, or proliferative, e.g. diabetic, retinopathy. It is well known that patients respond differently to chemical therapeutic medications. Therefore, inclusion of the compounds herein in the medical arsenal may provide relief to individuals who do not respond or respond poorly to other medications, such as chemotherapeutic treatments.

In addition, the compounds disclosed herein find utility in fertility control, for example as contraceptives.

For the treatment of skin diseases, such as psoriasis, topical application of the chemical(s) is the most direct method of use. For the treatment of internal tumors, for example implants of beads impregnated with the chemical(s) into tumors, or intravenous delivery are suitable options. To inhibit fertility, delivery to the oviduct can be accomplished, for example, by ingesting pills or by implanting the chemical under the skin, as has been done with other contraceptives, and discussed above.

In addition, the compounds of the present invention can be specifically targeted to the organs of interest. For example, they can be packaged in liposomes containing antibodies to cell surface receptors on target cells/organs.

Articles of Manufacture

The invention includes articles of manufacture comprising a compound of formula I, Ia, Ib, or Ic, or a pharmaceutically acceptable salt thereof, in a container, which has a label attached to it and/or contains a package insert. Suitable containers include, for example, bottles, vials, syringes, and test tubes. The containers may be formed from a variety of materials such as glass or plastic. The container holds a composition which is effective for treating the condition and may have a sterile access port (for example the container may be an intravenous solution bag or a vial having a stopper pierceable by a hypodermic injection needle). The label on, or associated with, the container, or the package insert indicates that the composition is used for treating the condition of choice. The article of manufacture may further comprise a second container comprising a pharmaceutically-acceptable buffer, such as phosphate-buffered saline, Ringer's solution and dextrose solution. It may further include other materials desirable from a commercial and user standpoint, including other buffers, diluents, filters, needles, syringes, and package inserts with instructions for use.

Further details of the invention will be apparent from the following non-limiting Examples.

### EXAMPLE 1

Identification of Compounds in Cigarette Smoke Solution that Inhibit Cell Proliferation and Angiogenesis

Materials and Methods

A. Media and Reagents

All buffers, including phosphate buffered saline (PBS), Earle's Balanced Salt Solution (EBSS), and cacodylate were made using Bamstead/Thermolyne nanopure water (Fisher Scientific, Tustin, CA). EBSS was made fresh daily from a 10× stock solution. To a 1× salt solution, sodium bicarbonate and HEPES were added to make (EBSS-H) (Talbot et al., Biol. Reprod. 58:1047-1053 (1998)). The pH of EBSS-H was adjusted to 7.4 with NaOH and was used as the control solution and to dilute test chemicals in all experiments. Glutaraldehyde (50%) was purchased from Electron Microscopy Supplies (Fort Washington, Pa.) and diluted to 3% in 0.1 M cacodylate buffer (pH 7.4) for use in fixation. Pyridine was purchased from VWR Scientific (San Francisco, Calif.) 2-methylpyridine, 3-methylpyridine, 2,3-dimethylpyridine, 2,4-dimethylpyridine, 2,4,6-trimethylpyridine, 2-ethylpyridine, 3-ethylpyridine, and 4-ethynylpyridine were purchased from Sigma-Aldrich Incorporated (Milwaukee, Wis.). 4,4-bipyridine and 3-(1-methylpyrrol-2-yl)pyridine (β-nicotyrine) were purchased from City Chemicals (West Haven, Conn.). 1-methyl-2-(3-pyridyl)pyrrolidine (nicotine) was purchased from Sigma (St. Louis, Mo.). 3-(1-pyrrolidin-2-yl)pyridine (nornicotine) was purchased from Toronto Research Chemicals (North York Ontario, Canada).

B. Solid Phase Extraction of Smoke Solutions

Solid Phase Extraction (SPE) cartridges were used to pre-clean smoke solutions and concentrate chemicals that inhibit cell proliferation and angiogenesis. All SPE cartridges were Bond Elut cartridges 3 cc w/500 g capacity purchased from Phenomex Torrance, Calif. Cartridges which were screened for their ability to bind inhibitory chemicals included a variety of non-polar, polar, and anion and cation exchange cartridges: NH2, 1OH, CN, CBA, SCX, SAX, C18, C8, C2, CH, SO, and PH.

The SPE cartridges were activated with 50 ml of HPLC grade methanol (Fisher Scientific), and then rinsed with 200 ml of deionized water. A column control was collected to assure that the methanol has been rinsed from the cartridge and that any toxic effect observed was solely due to the smoke solution. The smoke solution was placed through the cartridge and the “pass through” was collected and tested in the bioassays. The cartridge was then either eluted immediately or frozen at −20° C. for later analysis. SPE cartridges were eluted with 1 ml of HPLC grade methanol and the eluent was collected. The eluent was used in the GC/MS analysis and the biological assays. For the biological assays, the methanol was evaporated under nitrogen, and the residue was re-suspended in EBSS-HA (EBSS-H enriched with 1% BSA). A methanol control was used to assure that the observed inhibitory activity was not due to residual methanol in the sample.

C. Gas Chromatography-Mass Spectrometry (GC-MS)

A preliminary screen of solid phase extraction cartridges has shown that the C2 cartridge retained most of the inhibitory activity in the cell proliferation and angiogenesis assays. To identify the components in aqueous smoke solutions that inhibit cell proliferation and angiogenesis, mainstream and sidestream smoke solutions were analyzed with GC-MS after solid phase extraction on a C2. The equipment used was a Hewlett Packard 5890 GC interfaced to an HP-5970 MSD (quadropole mass selective detector) with a Zebron ZB1701 cyanopropyl phenyl column 30 m×0.32 mm and with a 1 μm phase thickness (Phenomenex, Torrance, Calif.). Helium was used as the carrier gas. The mass range examined was 40 to 350 with a threshold of 1500 with a sampling of 2.3 scans per second. The temperature program was an initial temperature of 45° C. for one minute with an increase of 10° C. per minute to a final temperature of 280° C. after ten minutes. The total run time was 34.5 minutes. Two microliters of the eluent sample were injected directly into the GC using a Hamilton gas-tight syringe. Identifications of compounds were made using the mass spectrometry data matched to library entries. Compound identities were confirmed using purified standards matched by mass spectrum and retention time.

D. Chick Chorioallantoic Membrane (CAM) Assay

The chick chorioallantoic membrane (CAM) assay is perhaps the most widely used assay for screening anti-angiogenic or growth inhibitory compounds (see, e.g. Ribatti et al., Int. J. Dev. Biol. 40:1189-1197 (1996)). Cell proliferation and angiogenesis were evaluated by measuring CAM area and BrdU labeling using the CAM assay as modified by Melkonian et al., J. Exp. Zool. (2001), in review. Fertilized chicken eggs were purchased from Hy-Line International (Lakeview, CA), then incubated at 37° C. at 85-90% relative humidity throughout the experiment. Experiments were done to evaluate cell proliferation, angiogenesis, and vascularization in CAMs between days 5 and 6 after fertilization. Albumin (2.5-3.0 cc) was removed on the fourth day after fertilization to drop the embryo away from the shell. Windows were then made in shells, and sealed with transparent tape to allow subsequent access to the CAM for experimental treatment. 200 μl of either EBSS-H or different concentrations of pure chemicals were added to the surface of each CAM at 10 AM on the fifth day after fertilization. A sham control group in which the window was opened and resealed was also included in each experiment. In all cases, the following five concentrations of test reagents were used, 5×10⁻³ M, 5×10⁻⁵ M, 5×10⁻⁷M, 5×10⁻⁹ M and 5×10⁻¹¹M. For 2-ethylpyridine and 3-ethylpyridine additional concentrations of 5×10⁻¹²M, 5×10⁻¹³M, 5×10⁻¹⁴ M and 5×10⁻¹⁵ M were used. For nicotine and nornicotine, concentrations of 5×10⁻⁴ M, 5×10⁻⁶ M, 5×10⁻⁸ M and 5×10⁻¹⁰ M were also used. The chemicals tested and their corresponding concentrations are summarized in Tables 1 and 2 below. Development was terminated at 10 AM on the sixth day after fertilization by fixing the embryo and the CAM with 3% glutaraldehyde in 0.1M cacodylate buffer (pH 7.4) for 2 hours at room temperature. A small volume of fixative was also injected under the CAM. The CAMs were later dissected from eggs, post-fixed in the same fixative for 24 hours at room temperature, then thoroughly rinsed in PBS.

E. Quantification and Imaging of CAMs

At least 6 CAMs were used in each control and experimental group. Parameters which measure aspects of cell proliferation, angiogenesis, and vacularization were quantified for each CAM. These parameters include: (1) for cell proliferation—CAM area, BrdU labeling of nuclei; (2) for angiogenesis—blood vessel pattern, blood vessel area, blood vessel diameter, and blood vessel branching; (3) for vascularization—density of angiogenic clusters. The area of each CAM was measured by placing the CAMs in PBS in a Petri dish under a Wild-M5A dissecting microscope (Max ERB instrument Co., Burbank, CA), then measuring the longest and shortest length of each CAM with a ruler to the precision of 0.5 mm. The data were recorded into Excel spreadsheet. CAM area was calculated using the formula:

Area=(½ A)×(½ B)×(π)

where A=longest length, B=longest width, π=3.14.

Means and standard deviations were then calculated for each control and treatment group. The treated group means were used to compare with control means to determine the effect of each chemical on CAM growth.

The surface of each CAM was further dissected out of the CAM pocket in PBS. CAMs were then flattened with 18×18−1.5 Fisherbrand microscope cover slip (Fisher Scientific, Pittsburgh, Pa.) in Fisherbrand 60×15 mm standard sterile polystyrene petri dish (Fisher Scientific, Pittsburgh, Pa.). Video images of each CAM were captured at 3× magnification using a Hitachi KP-D50U camera (Hitachi Inc, Torrance, Calif.) and stored on zip disks until further analysis.

Pattern formation was evaluated blindly using video image montages projected on a computer monitor. Images were ranked on a scale of 0 to 3, with 0 being similar to control CAMs and 3 being most dissimilar from controls.

The area occupied by major blood vessels in control and chemically treated CAMs was measured in digitalized images projected on a computer monitor using NIH image. Boxes (2.4 c 1.98 mm) were placed in each corner of each CAM image, which had been processed to visualize the major blood vessels. The percentage of pixels occupied by the blood vessels was quantified in each box and an average percentage was computed from the four boxes measured from each CAM. These values were used to compute means and standard deviations for each group.

Blood vessel diameter and branching were measured in images projected on a computer monitor using NIH Image software.

Density of angiogenic clusters was evaluated in toluidine blue stained histological sections that were digitalized and projected on a computer monitor.

F. BrdU Labeling of CAMs

Eight hours after the initial EBSS-H or chemical treatment on day 5 after fertilization, 1001L of 1 nM BrdU (Sigma, St. Louis, Mo.) were added to each CAM surface. Another 55 minutes after the BrdU treatment, the development of the CAMs was terminated by transferring the embryos and CAMs into a −20° C. freezer for 2-3 minutes. Embryos and CAMs were then fixed at −20° C. in 70% ethanol containing 50 mM glycine buffer, pH 2.0 for 1.5 hours. CAMs were then dissected out of the eggs, washed in PBS and dehydrated in 30%, 50%, 75%, 90% and 100% ethanol series. Dehydrated CAM tissues were further processed in Hypercenter II Tissue Processing System (Shandon Inc., Pittsburgh, Pa.), then embedded in paraffin using Sakura Tissue-TE (Shandon Inc., Pittsburgh, Pa.). Paraffin Sections were cut using a S20 Spencer microtome (Labequip, American Optical Corp., Ontario, Canada), and were mounted on Superfrost/plus glass slides (Fisher Scientific, Tustin, CA). Sections were deparaffinized with 3 changes of Hemo-De for 15 minutes each, 2 changes of 100% ethanol for 5 minutes each, then rehydrated in PBS. Paraffin sections were incubated in 1 N HCl for 1 hour at 40° C., washed in PBS for 10-15 minutes, then incubated in 1% sodium borohydride (Sigma, St. Louis, Mo.) for 10 minutes, and washed again in PBS. Sections were first incubated in anti-BrdU (1/100 dilution in PBS containing 1% BSA) for 2 hours at room temperature. Slides were then washed in PBS containing 0.1% BSA for 45-60 minutes, then incubated in secondary antibody (Alexa anti-mouse IgG, 1/100 dilution) for 1 hour at room temperature. Slides were washed with PBS plus 0.1% BSA for 45 minutes, and cover slides were mounted with Vectashield. BrdU and antibody controls were performed on both control and treated CAMs. Controls consisted of CAMs that were not exposed to BrdU and CAMs exposed to secondary antibody only. Slides were examined using a Zeiss photomicroscope with epifluorescence. Images were recorded using a Spot digital camera-SP401-115 (Diagnostic Instruments, Sterling Heights, MI) and processed and montaged using Adobe Photoshop (San Jose, Calif.).

G. Statistical Analyses

Group means of CAM and blood vessel area were compared by analysis of variance (ANOVA). When significance was found (p<0.05), either the Dunnett or Tukey-Kramer post hoc test was used to identify significantly affected groups. Dunnett's test compares treated group means to the control group. The Tukey-Kramer test compares mean values of each selected concentration with each other. Analyses were done using GraphPad (Instat, San Diego, Calif.). Data were checked to determine if they satisfied the assumptions of ANOVA. If the assumptions were not satisfied, the Kruskal-Wallis non-parametric test was used followed by Dunn's post hoc test.

Results:

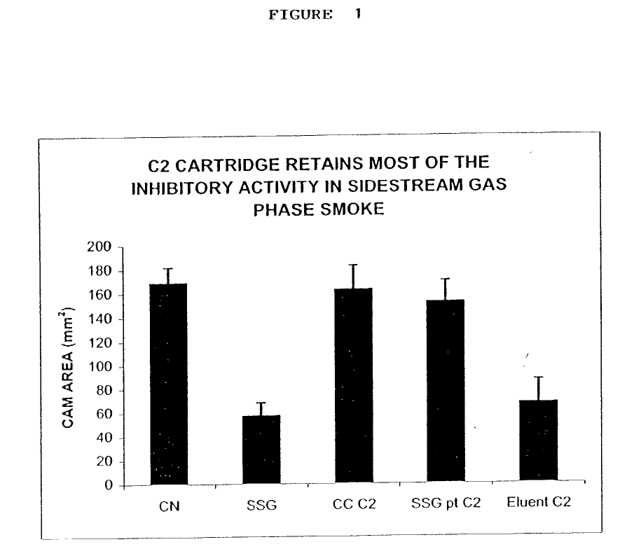

Identification of inhibitory chemicals in smoke solution: A preliminary screen of solid phase extraction cartridges showed that C2 solid phase extraction cartridges retained most of the chemicals in sidestream smoke solutions that inhibit CAM growth (FIG. 1). As noted in the figure legend, SSG pt C2 contains the chemicals in SSG (positive control) phase smoke that pass directly through the C2 cartridge. These chemicals did not significantly inhibit CAM growth. The eluent C2 group contains the chemicals that eluted off the C2 column after the SSG pass through had been collected. The eluent C2 group contains most of the inhibitory activity.

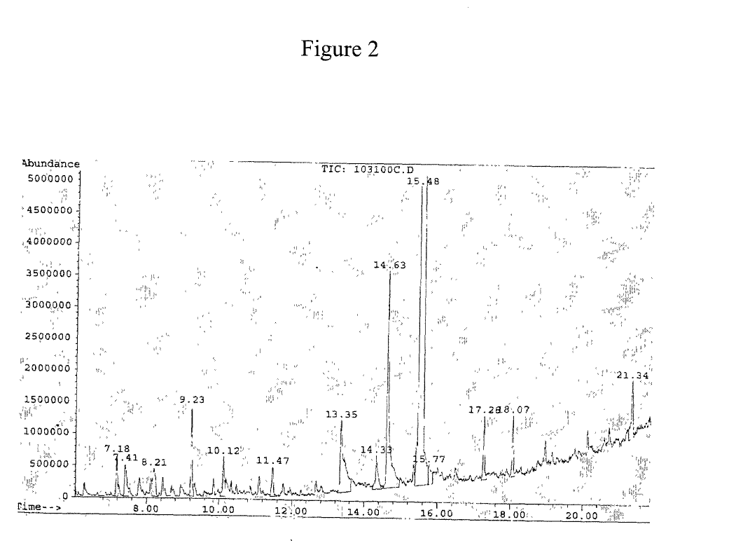

To determine what chemicals are retained by the C2 cartridge, 15 ml of sidestream smoke solution were passed through C2 cartridges which were then eluted with 1 ml of methanol to remove the inhibitory chemicals. Each eluent was concentrated in an evaporating disk, and the concentrated “pre-cleaned” eluent (1 μL) was injected into the GC-MS for chemical identification. A representative gas chromatogram of C2 eluent from a sidestream whole smoke solution is shown in FIG. 2. Using mass spectrometry, 12 pyridine derivatives were identified and tested at various concentrations for their effects on CAM area, which is a measure of CAM growth (Table 1). Cell proliferation was assayed by examining BrdU incorporation to into the nuclei, which is a measure of DNA synthesis.

1. Pyridine Derivatives Affected CAM Area.

On the fifth day after fertilization, either EBSS-H or different concentrations of test chemicals were added to each CAM. A sham group in which windows were opened and resealed without adding any solution was also included to determine if the vehicle EBSS-H had any effect on CAM area. Development was terminated 24 hours later, and CAMs were measured as described in Materials and Methods.

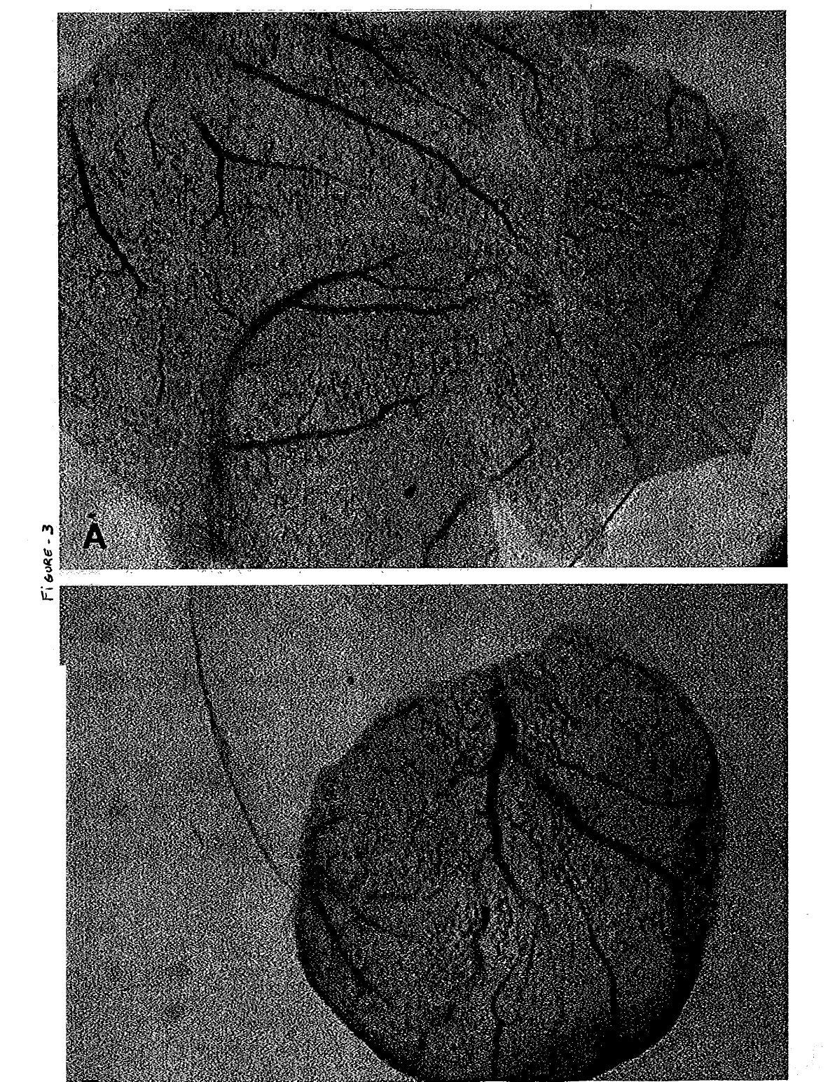

Macroscopic examination of day 6 CAMs revealed that at some doses, CAMs treated with the 12 pyridine derivatives were smaller than control CAMs. Representative video images of a control and 2-ethylpyridine treated CAM are shown at day 5.3 in FIG. 3. CAMs treated with the dilution buffer, EBSS-H were similar in size and appearance to the untreated controls. However, CAMs treated with 5e-3M 2-ethylpridine were significantly smaller than either control group and showed disturbance in major blood vessel pattern formation.

2. The Relationship Between Effective Doses and the Functional Groups Attached to the Pyridine Ring.

Simple Pyridine Rings Affected CAM Area at High Doses.

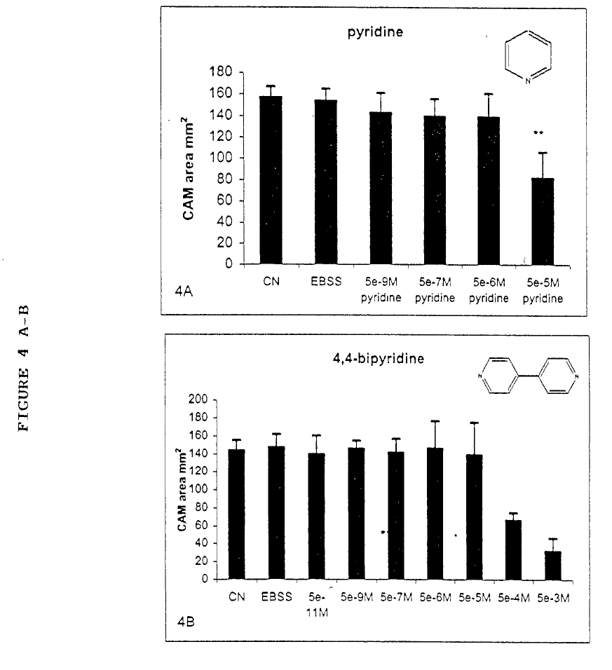

To quantify and compare the efficacy of the test compounds, CAM areas were measured for each treatment group. To cross compare the relative toxicity of various functional group substitutions, pyridine, although not present in C2 eluent of sidestream smoke solution, was first evaluated on day 5-6 CAMs (FIG. 4A). Treatment of CAMs with EBSS-H showed no effect on CAM area when compared to the untreated control (FIGS. 4A, 4B compare CN with EBSS-H). Concentrations of pyridine between 5e-9M to 5e-6M did not significantly affect CAM area when compared to the control and EBSS-H groups. However, at 5e-5M (3.96e-3 mg/ml) pyridine, CAM area was significantly less than either control group.

Using the effective concentration of simple pyridine ring as a comparative reference, the 12 pyridine derivatives identified in sidestream smoke solution were then tested for their effects on CAM area. 4,4-bipyridine was first tested to determine the effect of dimerization of 2 pyridine rings at 4,4 position on CAM area. The experiment with 4,4-bipyridine showed that addition of a second pyridine ring at position 4 of the pyridine increased the lowest effective dose to 5e-4M (7.81e-2 mg/ml) (FIG. 4B).

Single Methyl Substitutions on Pyridine Enhanced the Inhibitory Effect by 10 Thousand Times.

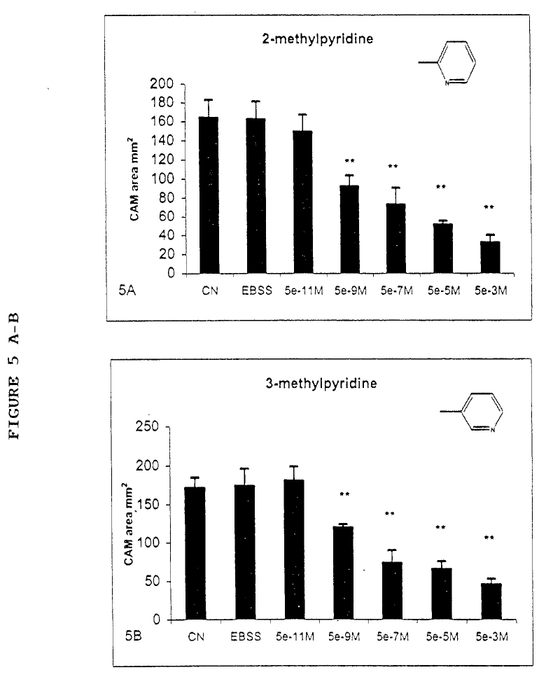

In the list of pyridine derivatives identified in sidestream smoke solution, two compounds have single methyl substitution. 2-methylpyridine and 3-methylpyridine were tested on day 5-6 CAMs to determine the effect of single methyl substitution on pyridine ring. Treatment of CAMs with EBSS-H had no effect on CAM area when compared to the untreated control (FIGS. 5A, 5B, compare CN with EBSS-H). However, CAM area was significantly decreased in a dose dependent manner with addition of a single methyl group at either position 2 or 3 of the pyridine ring (FIGS. 5A, 5B). Both 2-methylpyridine and 3-methylpyridine were effective at doses as low as 5e-9M (4.66e-7 mg/ml).

Double or Triple Methyl Substitutions to the Pyridine Ring did not Enhance the Inhibitory Effect.

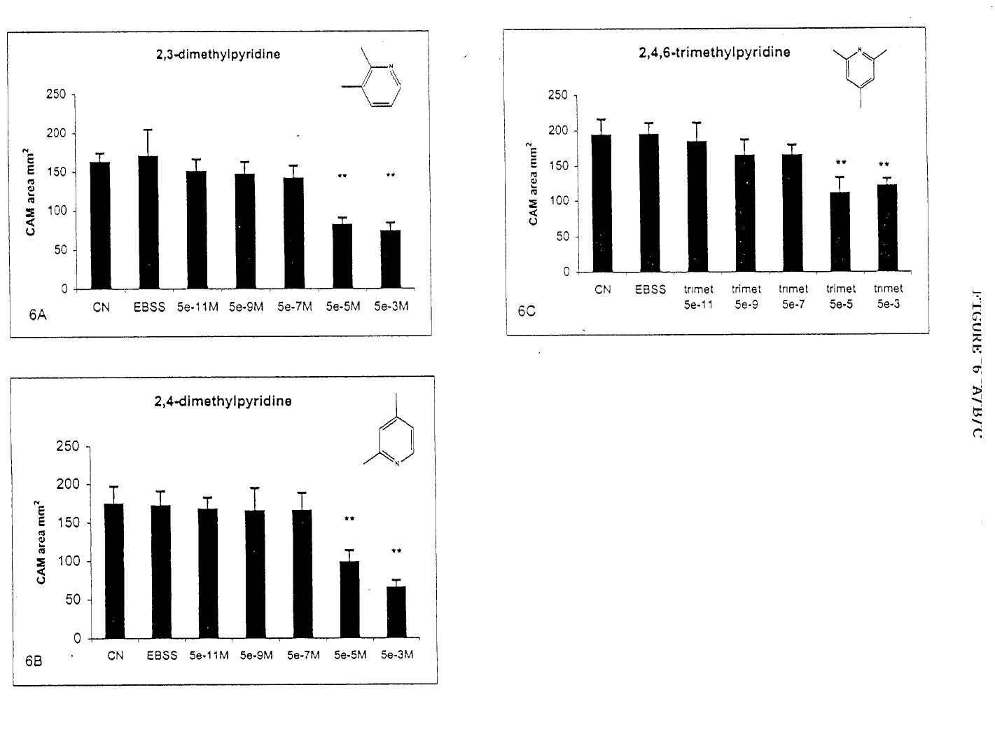

In the list of pyridine derivatives identified in sidestream smoke solution, three compounds have either double or triple methyl substitutions. 2,3-dimethylpyridine, 2,4-dimethylpyridine and 2,4,6-trimethylpyridine were tested on day 5-6 CAMs to determine the effect of multiple methyl substitutions on pyridine ring. Treatment of CAMs with EBSS-H had no effect on CAM area when compared to the untreated control (FIGS. 6A, 6B and 6C, compare CN with EBSS-H). The lowest effective dose for 2,3-dimethlypyridine and 2,4-dimethlypyridine was 5e-5M (5.36e-3 mg/ml). Addition of a third methyl group did not change the lowest effective dose of 2,4,6-trimethylpyridine (5e-5M, 6.10e-3 mg/ml). Neither double nor triple methyl substitutions affected CAM area when compared to the lowest effective dose of pyridine (5e-5M).

A Single Ethyl Substitution to the Pyridine Ring Enhanced the Inhibitory Effect by 10 Million Times.

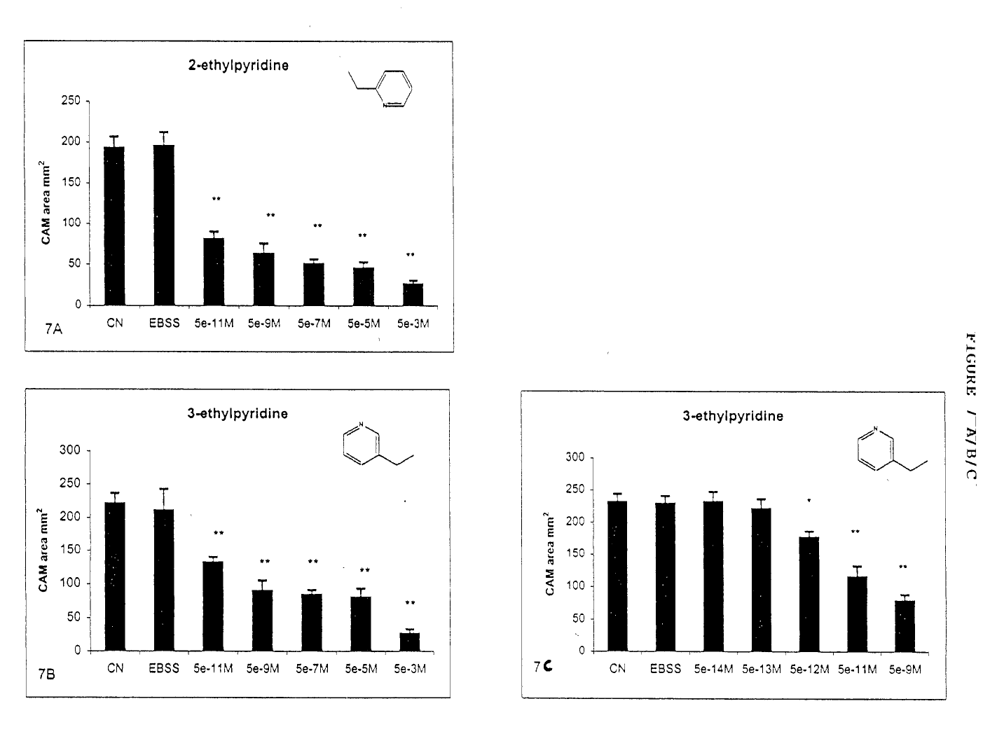

In the list of pyridine derivatives identified in sidestream smoke solution, two compounds have a single ethyl substitution. 2-ethylpyridine and 3-ethylpyridine were tested on day 5-6 CAMs to determine the effect of single ethyl substitution on pyridine ring. Treatment of CAMs with EBSS-H had no effect on CAM area when compared to the untreated control (FIGS. 7A, 7B, compare CN with EBSS-H). Substitution of a single ethyl group at either position 2 or 3 of the pyridine ring decreased the lowest effective dose to at least 5e-11M, a concentration that was an ineffective for all other chemicals tested (FIGS. 7A, 7B). Since all doses were highly inhibitory in this set of experiments, a second set of experiments was performed over the concentration range of 5e-9M to 5e-14M to define the lowest effective dose (FIGS. 7C, 7D). For both 2-ethylpyridine and 3-ethylpyridine, the lowest effective dose was 5e-12M (5.36e-10 mg/ml), which is 10 million times lower than the lowest effective dose of the single pyridine ring.

A Single Ethenyl Substitution to the Pyridine Ring did not Enhance the Inhibitory Effect.

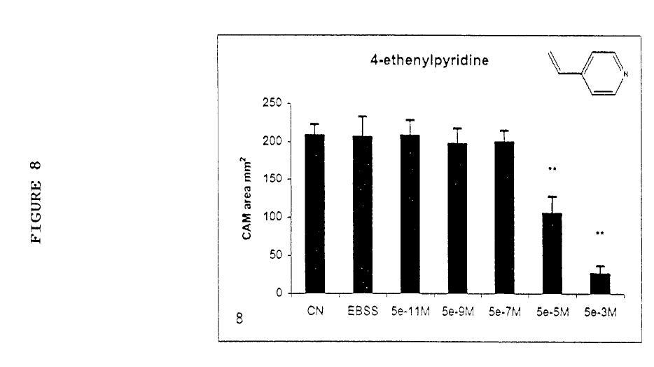

In the list of pyridine derivatives identified in sidestream smoke solution, one compound, 3-ethenylpyridine has a single ethenyl substitution. However, 3-ethenylpyridine is neither commercially available nor readily synthesized, therefore, its isomer, 4-ethenylpyridine which is commonly used as its substitute, was then tested on day 5-6 CAMs to determine the effect of single ethenyl substitution on pyridine ring. Treatment of CAMs with EBSS-H had no effect on CAM area when compared to the untreated control (FIG. 8 compare CN with EBSS-H). The lowest effective dose for 4-ethenylpyridine was 5e-5M (5.26e-3 mg/ml), which was equivalent to that of the pyridine ring. Addition of a double bond to the ethyl functional group created an ethenyl substitution and completely reversed the highly inhibitory effect of 3-ethylpyridine.

Substitution of Various Pyrrol Functional Groups had a Bimodal Effect on CAM Area.

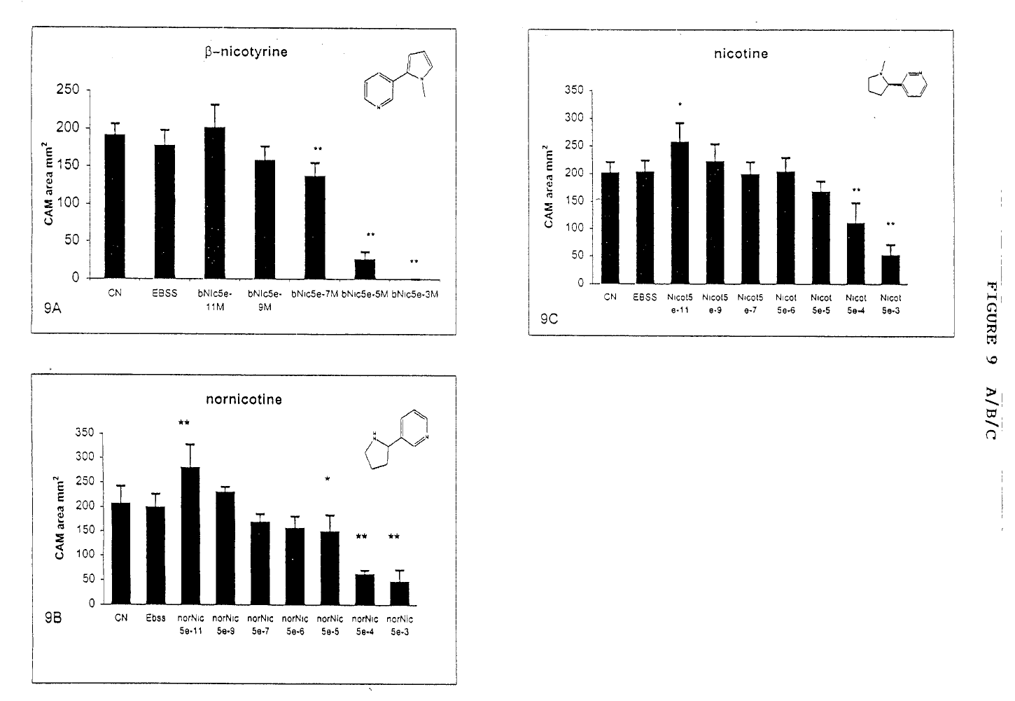

In the list of pyridine derivatives identified in sidestream smoke solution, three compounds have various pyrrol substitutions. 3-(1-methylpyrrol-2-yl)pyridine (β-nicotyrine), 3-(1-pyrrolidin-2-yl)pyridine (nornicotine), and 1-methyl-2-(3-pyridyl)pyrrolidine (nicotine) were tested on day 5-6 CAMs. Treatment of CAMs with EBSS-H had no effect on CAM area when compared to the untreated control (FIGS. 9A, 9B, 9C compare CN with EBSS-H). However, treatment of CAMs with this group of pyridine derivatives showed a bimodal effect, in which CAM area was decreased at high concentrations and was increased at low concentrations. The lowest effective inhibitory doses after the additions of 1-methylpyrrol-2-yl for β-nicotyrine, 1-pyrrolidin-2-yl for nornicotine, and 1-methyl-2-pyrrolidine for nicotine to the simple pyridine ring were 5e-7M (7.90e-5 mg/ml), 5e-5M (7.40e-3 mg/ml) and 5e-4M (8.10e-2 mg/ml), respectively. The various pyrrol additions caused moderate changes in lowest effective inhibitory doses compared to 5e-5M of simple pyridine ring. Unlike all other pyridine derivatives tested, a significant stimulatory effect was observed for 3-(1-pyrrolidin-2-yl)pyridine (nornicotine), and 1-methyl-2-(3-pyridyl)pyrrolidine (nicotine) substitutions at 5e-11M. There was also a slight increase for the 3-(1-methylpyrrol-2-yl)pyridine (β-nicotyrine) substitution, which was not significantly different from the controls.

A summary of the concentrations at which there was no observed adverse effect (NOAEL=No Observed Adverse Effect Level), and the lowest concentrations at which adverse effect was observed (LOAEL =Lowest Observed Adverse Effect Level) is provided for all compounds in Table 2 below.

3. Effect of 2-ethylpyridine and 3-(1-pyrrolidin-2-yl)pyridine (nornicotine) on Cell Proliferation as Measured by BrdU Labeling.

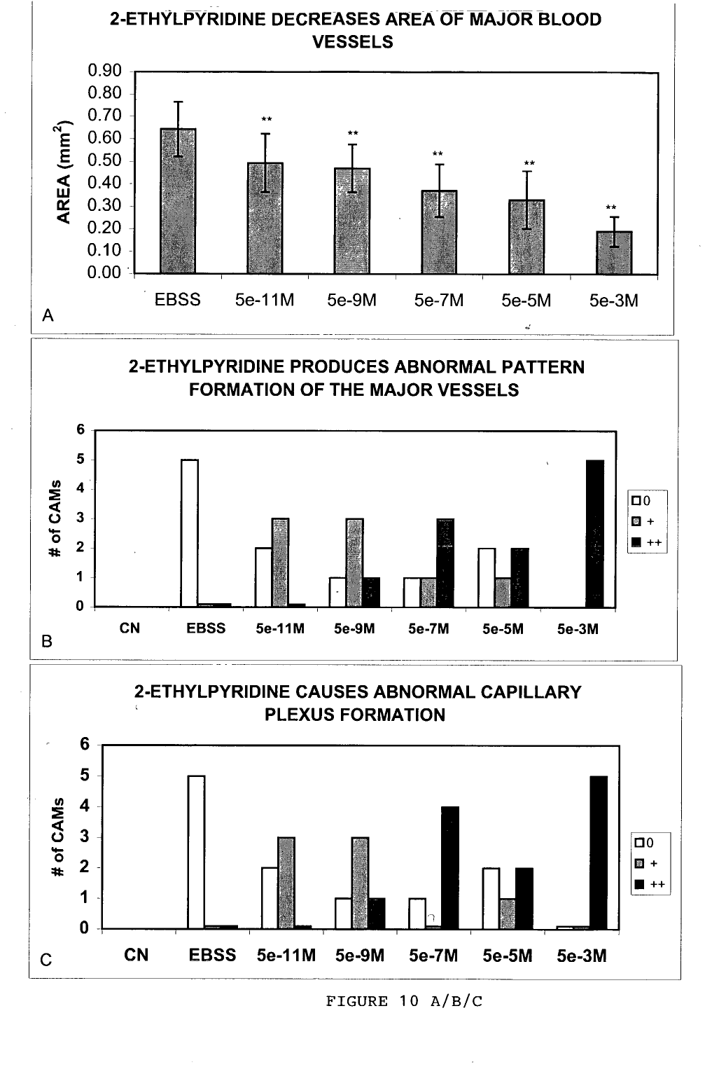

Previous work has shown that treatment of CAMs with mainstream or sidestream smoke solutions significantly inhibited incorporation of BrdU into nuclei of the ectoderm, endoderm, mesenchyme, and vasculature of day 5 CAMs (Melkonian et al, in preparation). To determine if pyridine derivatives similarly affect cell proliferation, day 5 CAMs were exposed to 5e-11 2-ethylpyridine or 5e-9 2methylpyridine for eight hours, then CAMs were labeled with BrdU. Treated CAMs showed significantly less incorporation of BrdU into nuclei than the untreated or EBSS treated controls (FIG. 10).

TABLE 1
The chemicals tested on CAMs and their corresponding concentrations.

| | | | | | | Concentration Tested | | | | | | |
|---|---|---|---|---|---|---|---|---|---|---|---|---|
| Chemical Tested | 5e−3M | 5e−4M | 5e−5M | 5e−6M | 5e−7M | 5e−8M | 5e−9M | 5e−10M | 5e−11M | 5e−12M | 5e−13M | 5e−14M |
| 2-ethylpyridine | X | | X | | X | | X | | X | X | X | X |
| 3-ethylpyridine | X | | X | | X | | X | | X | X | X | X |
| 2-methylpyridine | X | | X | | X | | X | | X | | | |
| 3-methylpyridine | X | | X | | X | | X | | X | | | |
| 2,3-dimethylpyridine | X | | X | | X | | X | | X | | | |
| 2,4-dimethylpyridine | X | | X | | X | | X | | X | | | |
| 2,4,6-trimethylpyridine | X | | X | | X | | X | | X | | | |
| nicotine | X | X | X | X | X | X | X | X | X | | | |
| nornicotine | X | X | X | X | X | X | X | X | X | | | |
| b-nicotyrine | X | | X | | X | | X | | X | | | |
| 4,4-bipyridine | X | X | X | X | X | | X | | X | | | |
| 4-ethenylpyridine* | X | | X | | X | | X | | X | | | |
| pyridine** | | | X | X | X | | X | | | | | |

*4-ethenylpyridine is not normally found in sidestream whole solution. However, since 3-ethenylpyridine is not commercially available nor is it readily synthesized, its isomer, 4-ethenylpyridine is commonly used as a substitute.
**Pyridine is not among the list of 12 pyridine derivatives identified in C2 eluent. This compound is tested as a reference to compare the efficacy of various functional group substitutions.

TABLE 2
Summary of all chemicals tested.

| | NOAEL | LOAEL | LOAEL |
|---|---|---|---|
| Chemical Tested | (Molar) | (Molar) | (mg/ml) |
| 2-ethylpyridine | 5e−13M | 5e−12M | 5.36e−10 mg/ml |
| 3-ethylpyridine | 5e−13M | 5e−12M | 5.36e−10 mg/ml |
| 2-methylpyridine | 5e−11M | 5e−9M | 4.66e−7 mg/lml |
| 3-methylpyridine | 5e−11M | 5e−9M | 4.66e−7 mg/lml |
| b-nicotyrine | 5e−9M | 5e−7M | 7.90e−5 mg/ml |
| 4-ethenylpyridine | 5e−7M | 5e−5M | 5.26e−3 mg/ml |
| 2,3-dimethylpyridine | 5e−7M | 5e−5M | 5.36e−3 mg/ml |
| 2,4-dimethylpyridine | 5e−7M | 5e−5M | 5.36e−3 mg/ml |
| 2,4,6-trimethylpyridine | 5e−7M | 5e−5M | 6.10e−3 mg/ml |
| nornicotine | 5e−6M | 5e−5M | 7.40e−3 mg/ml |
| Pyridine | 5e−7M | 5e−5M | 3.96e−3 mg/ml |
| 4,4-bipyridine | 5e−5M | 5e−4M | 7.81e−2 mg/ml |
| nicotine | 5e−5M | 5e−4M | 8.10e−2 mg/ml |

NOAEL = No Observed Adverse Effect Level
LOAEL = Lowest Observed Adverse Effect Level

4. Effect of 2-ethylpyridine on the Area of Major Blood Vessels

As shown in FIG. 10A, 2-ethylpyridine significantly decreased the area occupied by the major blood vessels in the CAM. At doses as low as 5e-11M blood vessel area was significantly decreased when compared to the EBSS control.

5. Effect of 2-ethylpyridine on Dendritic Branching Pattern

As shown in FIG. 10B, the normal dendritic branching pattern seen in control CAMs was disrupted by all doses of 2-ethylpyridine that were tested. 0=the pattern characteristic of controls. +=a significant difference in pattern formation from controls. ++=a highly significant difference in pattern formation from controls. The number of CAMs categorized as 0, +, or ++ is plotted for each dose tested.

6. Effect of 2-ethylpyridine on the Formation of Capillary Plexus

2-ethylpyridine significantly inhibited formation of the capillary plexus. Video images of CAMs were rated as 0, +, or ++ based on the amount of capillary plexus present in the controls or treated groups. All doses tested produced significant adverse effects on the formation of the plexus.

### EXAMPLE 2

Identification in Cigarette Smoke of Compounds that Inhibit Oviductal Functioning in Hamsters

It is known that the structure and function of the oviduct is adversely affected by solutions of mainstream and sidestream cigarette smoke (Magers et al., Reprod. Toxicol. 9:513-525 (1995)). It has also been reported that oocyte pickup rate and ciliary beat frequency are inhibited by mainstream (MS) and sidestream (SS) cigarette smoke solutions (Talbot et al., Biol. of Reprod. 58:1047-1053 (1998); Knoll et al., Biol. of Reprod. 53:29-37 (1995); Knoll et al., Reprod. Toxicol. 12(1):57-68 (1998)). To study the interaction of smoke with the oviduct experimentally, we developed an in vitro assay to measure oocyte pickup rate, ciliary beat frequency, and infundibular muscle contraction using hamsters (Huang et al., Molecular Reprod. Devel. 47:312-322 (1997)). The purposes of this study were to identify the individual toxicants in cigarette smoke solutions that affect oviductal functioning, to determine their effective doses, and to establish their mode of action.

Materials and Methods

A. Animals

Female golden hamsters (Mesocricetus auratus) purchased from Harlan Sprague Dawley (San Diego, Calif.), were maintained on a 14:10 light:dark cycle (6 AM to 8 PM light) in a room at 26° C. and food was administered ad libitum. Hamsters were cycled daily by checking for a vaginal discharge, which occurs on day 1 of their estrous cycle. Female golden hamsters were induced to ovulate by intraperitoneal injection with 25 International Units (i.u.) of human chorionic gonadotropin (hCG) (Sigma Chemical Co., St. Louis, Mo.) on day 3 of the estrous cycle and used twelve hours later on day 4 for all experiments. In some cases, hamsters were superovulated by administering 25 i.u. of pregnant mare's serum gonadotropin (PMSG) (CalBiochem, La Jolla, Calif.) at 10 a.m. on day 1 of their estrous cycle, followed by 25 i.u. of hCG on day 3. Unfertilized follicular oocytes were collected by puncturing mature tertiary follicles with an insect pin 12 hours after hCG administration. Unfertilized oviductal oocytes were collected in EBSS containing 0.1% BSA for use in the oocyte pick-up rate assay.

B. Media

Earle's Balance Salt Solution (EBSS) was made fresh daily from a 10× stock solution. To a 1× salt solution, sodium bicarbonate, and HEPES were added to make (EBSS-H), and the solution was enriched with 1% BSA to make (EBSS-HA) and used for dissection and incubation. EBSS-HA was used as the control solution for all experiments. The pH of the medium was adjusted to 7.4 with NaOH for all experiments. EBSS supplemented with bovine serum albumin (BSA) has been used in previous studies to create a pseudo-physiological environment (Norwood et al., Biol. of Reprod. 23:788-791 (1980); Huang et al., Molecular Reprod. Devel. 47:312-322 (1997); Knoll et al., Reprod. Toxicol 12(1):57-68 (1998); Talbot et al., Mol. Biol. Cell 10:5-8 (1999); Lam et al., Biol. of Reprod. 62:579-588 (2000)).

C. Preparation of Smoke Solutions

Smoke solutions were prepared using an University of Kentucky analytical smoking machine (Knoll et al., Reprod. Toxicol., 1998, supra). The set-up included a puffer box and a peristaltic pump, which pumped the smoke into a 10 ml solution of EBSS-H. Mainstream whole smoke solutions (MSW) were made from 60 puffs of mainstream smoke pushed through EBSS-H. Sidestream whole smoke solutions (SSW) were made by collecting the smoke that was produced from the burning end of the cigarette. Preparation of the sidestream smoke solution required two peristaltic pumps. One pump created the mainstream smoke which was exhausted to waste, the other pump collected the continuous stream of sidestream smoke from the burning end of the cigarette, which passed through the EBSS-H solution in the flask. Sidestream smoke was continuously pumped through the EBSS-H solution for the length of time equivalent to thirty puffs of mainstream smoke (Knoll et al 1998, supra).

D. Solid Phase Extraction of Smoke Solutions and Gas Chromatography-Mass Spectrometry (GC-MS)

Solid phase extraction of smoke solutions and GC-MS were performed as described in Example 1 above.

E. Purified Standards

Purified standards of the pyridine compounds identified using mass spectrometry were purchased and tested in dose-response studies to determine which chemicals adversely affected oviductal functioning. The following chemicals were purchased: 2,2-bipyridine, 4,4-bipyridine, and pyridine, 3-(1-methyl-1H-pyrrol-2-yl)-(common name: β-nicotyrine) from City Chemical LLC (West Haven, Conn.); 2,4,6-trimethylpyridine, 2-ethyl pyridine, 3-ethyl pyridine, 2,3-dimethylpyridine, 2,4-dimethylpyridine, 2,5-dimethylpyridine, 3,4-dimethylpyridine, 2-methylpyridine, and 3-methylpyridine from Aldrich (Milwaukee, Wis.); cotinine and nicotine from Sigma (St. Louis, Mo.); pyridine, 3-(3,4-dihydro-2H-pyrrol-5-yl)-(common name: myosmine) from Toronto Research Chemicals (North York Ontario, Canada); and pyridine from VWR (Brisbane, CA). Dilutions for the dose-response studies were made by weighing out 10-15 mg of the chemical and diluting it in 1 ml of EBSS to make a 10-15 mg/ml solution. Ten-fold serial dilutions were made by adding 10 μL of the chemical solution to 90 μL of EBSS.

F. Oocyte Pickup Rate (OPR) Assay

Hamsters were injected with 25 I.U. of human chorionic gonadotropin (hCG) on the evening of Day 4 of their estrous cycle. On the day of the experiment, approximately 12 hours after the injection of hCG, hamsters were sacrificed using CO₂, and oviducts and ovaries were removed. During dissection the oviduct and ovary were separated. Ovaries were isolated in EBSS-HA and expanded follicles were poked with a dissecting needle to release the oocyte cumulus complexes (OCCs). Oocyte cumulus complexes were transferred to a fresh Petri dish and placed in EBSS-HA. The infundibulum was cut from the oviduct leaving part of the ampulla attached to function as a handle. The infundibulum was placed into a perfusion chamber containing EBSS-HA (the control medium), the ampulla was placed into the holding pipette so the infundibulum remained stationary throughout the experiment.

Oocyte cumulus complex pick-up rate was measured using hamster oviduct explants in an in vitro assay (Huang et al., 1997, supra; Knoll et al., 1998, supra; Talbot et al., 1998, supra). The perfusion chambers containing the infundibular preparations were viewed at 25× using a Wild stereoscopic microscope (model BD-HF) containing an ocular micrometer. An oocyte cumulus complex was placed at the base of the infundibulum and the length of time needed for the complex to traverse a defined path to the ostium was recorded. This was the control value of oocyte pick-up rate. Then the test smoke solution or chemical was added to the perfusion chamber and allowed to incubate for five minutes after which pickup-rate was measured again. The perfusion chamber was washed out with EBSS and left to “recover” for five minutes. Oocyte pickup-rate was measured again and this rate was recorded as the “recovery” measurement. This sequence was repeated using increasing concentrations of test chemicals until all doses were tested.

G. Ciliary Beat Frequency Assay (CBS)

Ciliary beat frequency was measured using hamster oviduct explants in an in vitro assay (Knoll et al., 1998, supra; Talbot et al., 1998, supra). Perfusion chambers containing infundibula were placed on a Wild stereoscopic microscope (model BD-HF) and viewed at 50×. A video image was sent from the microscope to a Dell computer using a Hitachi camera and recorded using a VCR onto a Super VHS videotape. To measure cilia beat frequency the videotape was played back frame-by-frame. The VCR recorded 15 frames per second. Each frame represented a fraction of a cycle; the completion of a cycle was equivalent to one beat. Number of frames per cycle (or frames per beat) were measured and then converted into beats per second. Ten measurements were made for each infundibulum for each treatment group. Recordings were made for 40-60 seconds per region, which ensured an adequate amount of data for analysis.

H. Infundibular Muscle Contraction Assay

The infundibular muscle contraction assay was developed after observing consistent infundibular contractions that slowed or ceased after treatment with mainstream smoke solutions. Perfusion chambers containing infundibula were placed on a Wild stereoscopic microscope (model BD-HF) and viewed at 50×. A video image was sent from the microscope to a Dell computer using a Hitachi camera and the infundibular contractions could be observed and measured on the computer monitor. As the infundibulum contracted, the image shown on the monitor moved a measurable distance. This distance was measured during each contraction by placing a ruler against the computer monitor. These values (in centimeters) were then converted to actual distance by capturing an image of a ruler under the microscope and converting the measured values to micrometers. Frequency was also measured by observing the contracting infundibula on the computer monitor. The number of times the infundibulum contracted per minute was recorded. Each parameter was measured four to six times per infundibulum per treatment group. Preliminary data showed little to no variation between readings for a given treatment group.

I. Statistical Analysis

The statistical significance of the results were evaluated in the oocyte pick-up rate, ciliary beat frequency, and infundibular muscle contraction assays using one-way analysis of variance (ANOVA) with Instat (Graphpad, San Diego, Calif.). Means were considered to be significantly different for p<0.05. Post hoc comparisons were made using Dunnet's test. When data did not satisfy the assumptions of ANOVA, analyses were done using Kruskal-Wallis non-parametric test followed by Dunn's post hoc test for p<0.05.

Results

A. General Strategy

The mainstream whole smoke solution was found to inhibit oocyte pickup rate, ciliary beat frequency, and infundibular muscle contraction. To identify individual components in the smoke solution, solid phase extraction (SPE) cartridges were used to fractionate the smoke by removing certain classes of components. Each fraction was tested in the bioassay to determine toxicity. The fractions from the most effective SPE cartridges were injected into the GC-MS and individual compounds were identified. Purified chemicals of the identified compounds were purchased and tested in dose-response studies.

B. Initial Screen of Solid Phase Extraction Cartridges

Solutions of mainstream whole cigarette smoke were passed through a variety of polar and nonpolar solid phase extraction cartridges. The pass through solution was tested in the oocyte pickup rate, ciliary beat frequency, and infundibular contraction assays. Table 3 shows the results of the pass through solutions for each solid phase extraction cartridge for the three bioassays. The data are presented as a percent of control. A low value indicates high toxicity of the pass through solution. A high value indicates that the cartridge removed the toxicants and therefore the pass through showed little or no inhibition in the bioassays. Several cartridges (NH2, PH, C8, and CN) were very effective at removing the toxicants from the mainstream smoke solution that inhibited oocyte pickup rate, ciliary beat frequency, and infundibular muscle contraction (Table 3)

TABLE 3
Mainstream Whole Smoke Solution passed through Solid Phase
Extraction Cartridges and Tested in Bioassays

| | | | Infundibular Contraction | |
|---|---|---|---|---|
| Cartridge | OPR | CBF | Distance | Frequency |
| Nonpolar | | | | |
| CN | 92.6 | 107 | 100 | 100 |
| PH | 98.2 | 85.5 | 71.3 | 75 |
| C8 | 99.9 | 95.5 | 45 | 66.7 |
| CH | 63 | 113 | 12.5 | 50 |
| C18 | 43.3 | 55 | 100 | 80 |
| C2 | 42.5 | 67.8 | 36 | 66.7 |
| Polar | | | | |
| NH2 | 100 | 121 | 100 | 33.3 |
| SI | 49.7 | 132 | 100 | 40 |
| 2OH | 41.2 | 48.9 | 100 | 88.2 |
| CBA | 40.33 | 53.4 | 60 | 57 |
| SAX | 22 | 96 | 100 | 33.3 |
| SCX | 10.45 | 112 | 86 | 60 |

A low value indicates inhibition of oocyte pickup rate (OPR), ciliary beat frequency (CBF), or infundibular muscle contraction. A high value indicates retention of the toxicants by the cartridge and little or no inhibition in the bioassays.

C. GC-MS of Eluent of Each Cartridge and Pyridine Compounds Identified

TABLE 4
Pyridine Compounds Identified in Mainstream and Sidestream Smoke
Solutions after Solid Phase Extraction

| | m-NH2 | s-NH2 | m-C8 | s-C8 | m-PH | s-PH | m-CN | s-CN |
|---|---|---|---|---|---|---|---|---|
| 2-methyl pyridine | | | X | X | | | | X |
| 3-methyl pyridine | | X | X | X | X | X | X | X |
| 4-methyl pyridine | | X | | | X | | | X |
| 2,6 dimethyl pyridine | | | X | | | | | X |
| 2,3 dimethyl pyridine | | | | | | | | X |
| 3,4-dimethyl pyridine | | | | | | | | X |
| 2,5-dimethyl pyridine | | | | | | | X | |
| 2-ethyl pyridine | | | | X | | | | |
| 3-ethyl pyridine | | | | X | | | | X |
| 4-ethyl pyridine | | | | | | | | X |
| 3-ethenyl pyridine | | X | X | | | X | X | X |
| 2-ethyl, 6-methyl | | | | | | | X | X |
| pyridine | | | | | | | | |
| 4,4-bipyridine | X | | X | X | X | X | | X |
| nicotine | X | X | X | X | X | X | X | X |
| B-nicotyrine | X | X | X | X | X | X | X | X |
| myosmine | X | | X | X | X | X | | X |
| cotinine | X | | | | | X | | |
| pyridine, 3-(1- | | | | | | X | | |
| methyl-2- | | | | | | | | |
| pyrrolidinyl)- | | | | | | | | |

m = mainstream smoke through the solid phase extraction cartridge
s = sidestream smoke solution through the solid phase extraction cartridge

The NH2, C8, PH, and CN cartridges effectively removed the inhibitory components from mainstream smoke solutions when the eluents were tested in the oviductal assays. Therefore, the individual components of the smoke solutions were analyzed after extraction with these cartridges. The table shows the pyridine compounds identified with the different cartridges for solutions of mainstream and sidestream cigarette smoke.

D. Dose-Response Analysis of 3-ethylpyridine for Oocyte Pickup Rate, Ciliary Beat Frequency, and Infundibular Contraction

For the ciliary beat frequency, oocyte pickup rate, and infundibular muscle contraction graphs, purified 3-ethylpyridine was purchased and dissolved in EBSS. A hamster infundibulum was exposed to a dose range of 9.33 e-7 to 9.33 e-4 mM (four dose groups total) for five minutes and oocyte pickup rate, ciliary beat frequency, and infundibular contraction were measured. After each dose, the infundibulum was allowed to recover for twenty minutes and then a “recovery” measurement was made before the next dose was administered.

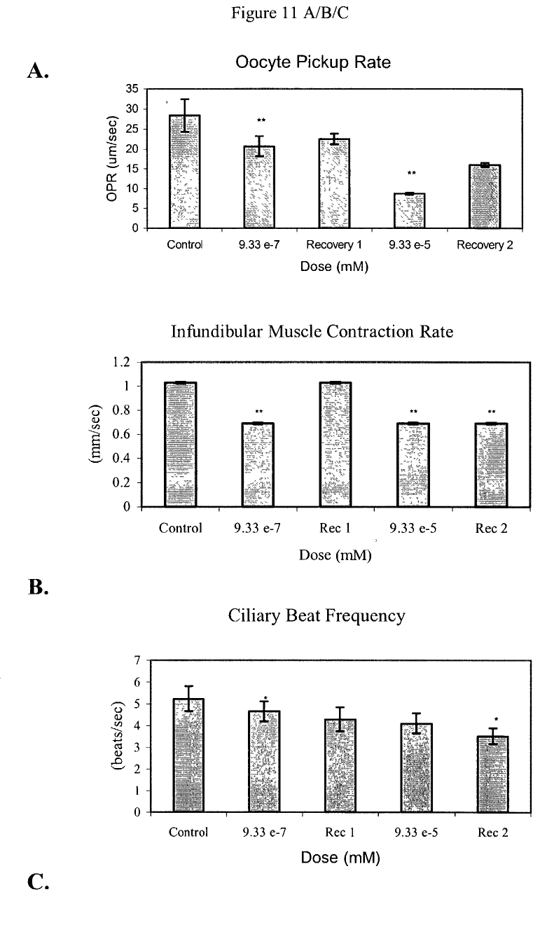

Two infundibula were used to test 3-ethylpyridine. Each infundibulum was exposed to 2 dose groups. Data for a single infundibulum are shown. Oocyte pickup rate (OPR), ciliary beat frequency (CBF), and infundibular muscle contraction rate were significantly inhibited at 9.33 e-7 mM of 3-ethylpyridine, ad shown in FIGS. 11A-C. The effect was partially reversible after the recovery period for OPR and contraction rate, but CBF continued to decrease during the recovery period.

E. Dose-Response Data for the Pyridine Compounds Identified and Tested

The dose-response data are shown in the following Table 5. The compounds are arranged from the lowest effective dose (top) that causes inhibition to oocyte pickup rate (OPR) to the highest effective dose (bottom). The compounds have similar trends in their effective doses causing inhibition to ciliary beat frequency and infundibular contraction.

TABLE 5
Dose-Response of Individual Cigarette Smoke Components
on the Hamster Oviduct In Vitro

| | | Oocyte Pickup | Ciliary Beat | Contraction |
|---|---|---|---|---|
| | Dose Range | Rate | Frequency | Rate |
| CHEMICAL | Tested (M) | Effective Dose (M) | Effective Dose (M) | Effective Dose (M |
| 2-ethyl pyridine | 10-13 to 10-11 | 9.35 × 10-12 | 9.35 × 10-11 | 9.35 × 10-13 |
| 4-vinyl pyridine | 10-11 to 10-8 | 1 × 10-11 | 1 × 10-9 | 1 × 10-11 |
| 2-methyl pyridine | 10-13 to 10-10 | 1.23 × 10-11 | N.D. | 1.23 × 10-11 |
| 3-ethyl pyridine | 10-12 to 10-9 | 9.33 × 10-10 | 9.33 × 10-9 | 9.33 × 10-10 |
| myosmine | 10-10 to 10-6 | 6.85 × 10-9 | 6.85 × 10-8 | 6.85 × 10-8 |
| B-nicotyrine | 10-10 to 10-7 | 6.33 × 10-9 | 6.30 × 10-8 | XXX |
| 2,4,6-trimethyl pyridine | 10-9 to 10-6 | 8.25 × 10-8 | 8.25 × 10-6 | 8.25 × 10-8 |
| 2,4-dimethyl pyridine | 10-10 to 10-7 | 9.34 × 10-7 | XXX | 9.34 × 10-9 |
| 2,3-dimethyl pyridine | 10-10 to 10-7 | 9.34 × 10-7 | 9.34 × 10-7 | 9.34 × 10-9 |
| 4,4-bipyridine | 10-7 to 10-3 | 8.78 × 10-6 | 8.78 × 10-7 | 8.78 × 10-4 |
| 3,4-dimethyl pyridine | 10-7 to 10-2 | 1.76 × 10-5 | XXX | 1.76 × 10-4 |
| 3-methyl pyridine | 10-7 to 10-1 | 1.23 × 10-5 | XXX | 1.23 × 10-2 |
| pyridine | 10-7 to 10-2 | 1.27 × 10-5 | 1.27 × 10-3 | 1.27 × 10-3 |
| cotinine | 10-7 to 10-2 | 2.84 × 10-5 | 2.84 × 10-5 | XXX |
| 2,5-dimethyl pyridine | 10-9 to 10-5 | >9.34 × 10-5 | XXX | >9.34 × 10-5 |
| 2,2-bipyridine | 10-5 to 10-2 | 8.74 × 10-4 | 8.74 × 10-3 | 8.74 × 10-2 |
| nicotine | 10-6 to 10-1 | 9 × 10-2 | XXX | 6.7 × 10-2 |
| phenol | 10-6 to 10-3 | 9.68 × 10-3 | 9.68 × 10-3 | 9.68 × 10-3 |

XXX = No significant effect at highest dose tested.
N.D. = Not Determined

All references cited throughout the disclosure and all references cited therein are hereby expressly incorporate by reference. While the present invention has been described herein with reference to specific embodiments thereof, it should be understood by those skilled in the art that various changes may be made and equivalents may be substituted without departing from the spirit and scope of the invention. In addition, many modifications may be made to adapt to a particular situation, material, composition of matter, process, to the objective, spirit and scope of the present invention. All such modifications are intended to be within the scope of the claims appended herein.

What is claimed is:

1. A method of inhibiting cell proliferation comprising contacting said cell with a growth inhibitory amount of a compound of formula I, 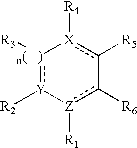
wherein
X, Y and Z independently represent C or N;
------ is an optional double bond;
n is 0 or 1;
R₁, R₂, and R₄ independently represent hydrogen, a chemical bond, C₁₋₁₀ alkyl; C₂₋₁₀ alkenyl; C₂₋₁₀ alkinyl; aryl; aryl-C₁₋₁₀ alkyl; C₃₋₁₀ heterocyclyl; C₅₋₁₀ heteroaryl; halo, CF₃; NO₂; NHC(O)R*, OR, said alkyl alkenyl, alkinyl, aryl, arylalkyl, heterocyclyl, or heteroaryl being optionally substituted;
R₃, R₅, and R₆ independently represent hydrogen, C₁₋₁₀alkyl; C₂₋₁₀ alkenyl; C₂₋₁₀ alkinyl; aryl; aryl-C₁₋₁₀alkyl; C₃₋₁₀ heterocyclyl; C₅₋₁₀ heteroaryl; halo, CF₃; NO₂; NHC(O)R*, OR, said alkyl, alkenyl, alkinyl, aryl, heterocyclyl, or heteroaryl being optionally substituted; or
R₅ and R₆ together form a 5- or 6-member aryl, heterocyclyl or heteroaryl group;
R is hydrogen or C₁₋₆ alkyl;
R* is hydrogen, or C₁₋₆ alkyl, or OH,
wherein the optional substituents are preferably selected from the group of one to three OH, C₁₋₆ alkyl, halo, NO₂, C₁₋₆ alkoxy, and CF₃,
or a pharmaceutically acceptable salt thereof.

2. The method of claim 1 wherein said cell is contacted with a compound of formula Ia, 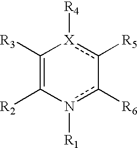
wherein
X is C or N;
------ is an optional double bond;
R₁ and R₄ independently represent hydrogen, a chemical bond, C₁₋₁₀alkyl; C₂₋₁₀ alkenyl; C₂₋₁₀ alkinyl; aryl; aryl-C₁₋₁₀alkyl; C₃₋₁₀ heterocyclyl; C₅₋₁₀ heteroaryl; halo, CF₃; NO₂; NHC(O)R*, OR, said alkyl, alkenyl, alkinyl, aryl, arylalkyl, heterocyclyl, or heteroaryl being optionally substituted;
R₂, R₃, R₅, and R₆ independently represent hydrogen, C₁₋₁₀alkyl; C₂₋₁₀ alkenyl; C₂₋₁₀ alkinyl; aryl; aryl-C₁₋₁₀alkyl; C₃₋₁₀ heterocyclyl; C₅₋₁₀ heteroaryl; halo, CF₃; NO₂; NHC(O)R*, OR, said alkyl, alkenyl, alkinyl, aryl, heterocyclyl, or heteroaryl being optionally substituted; or
R₅ and R₆ together form a 5- or 6-member aryl, heterocyclyl or heteroaryl group;
R is hydrogen or C₁₋₆ alkyl;
R* is hydrogen, or C₁₋₆ alkyl, or OH,
wherein the optional substituents are preferably selected from the group of one to three OH, C₁₋₆ alkyl, halo, NO₂, C₁₋₆ alkoxy, and CF₃,
or a pharmaceutically acceptable salt thereof.

3. The method of claim 2 wherein R₁ is a chemical bond; R₄ is hydrogen or a chemical bond; R₂ and R₃ independently represent hydrogen, C₁₋₁₀alkyl; C₂₋₁₀ alkenyl; or C₂₋₁₀ alkinyl; the dotted lines represent a double bond; and one of R₅ and R₆ is a heterocyclyl or heteroaryl ring, and the other is hydrogen.

4. The method of claim 2 wherein X is C; R₁ is a chemical bond, the dotted lines represent a double bond; R₄ is a heterocyclyl or heteroaryl ring; and R₂, R₃, R₅, and R₆ each independently is hydrogen, C₁₋₁₀alkyl; C₂₋₁₀ alkenyl; or C₂₋₁₀ alkinyl.

5. The method of claim 4 wherein R₄ is a heteroaryl ring.

6. The method of claim 5 wherein R₄ is pyridyl.

7. The method of claim 2 wherein said cell is contacted with a compound of the formula Ib 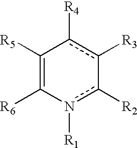
wherein R₁, R₂, R₃, R₄, R₅, and R₆ are as defined in claim 2.

8. The method of claim 7 wherein R₁ is a chemical bond; one, two, or three of R₂, R₃, R⁴, R₅, and R₆ are C₁₋₁₀alkyl; C₂₋₁₀ alkenyl; or C₂₋₁₀ alkinyl, and the others are hydrogen.

9. The method of claim 7 wherein R₁ is a chemical bond; one of R₂, R₃, and R₄ is a heterocyclyl or heteroaryl ring, while the others are hydrogen.

10. The method of claim 9 wherein R₅, and R₆ are both hydrogen.

11. The method of claim 2 wherein said cell is contacted with a compound of formula Ic 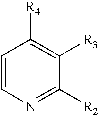
wherein R₂, R₃, and R₄ are as defined in claim 2.

12. The method of claim 11 wherein at least one of R₂, R₃, and R₄ is C₁₋₁₀alkyl; C₂₋₁₀ alkenyl; or C₂₋₁₀ alkinyl, and the others are hydrogen.

13. The method of claim 1 wherein said cell is a tumor cell.

14. The method of claim 13 wherein said tumor is a cancer.

15. The method of claim 14 wherein said cancer is selected from the group consisting of breast cancer, prostate cancer, colon cancer, squamous cell cancer, small-cell lung cancer, non-small cell lung cancer, gastrointestinal cancer, pancreatic cancer, cervical cancer, ovarian cancer, liver cancer, bladder cancer, colorectal cancer, endometrial carcinoma, kidney cancer, thyroid cancer, and hepatic carcinoma.

16. The method of claim 1 wherein said cell is an epithelial cell.

17. The method of claim 1 wherein said growth inhibitory amount is in the femtomolar range.

18. The method of claim 1 wherein said growth inhibitory amount is in the nanomolar range.

19. The method of claim 1 wherein said growth inhibitory amount is in the range of 10 to 100 molecules per cell.

20. A method of inhibiting angiogenesis in a cell, comprising contacting said cell with an effective amount of a compound of formula I 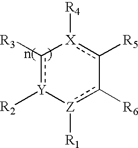
wherein
X, Y and Z independently represent C or N;
------ is an optional double bond;
n is 0 or 1;
R₁, R₂, and R₄ independently represent hydrogen, a chemical bond, C₁₋₁₀ alkyl; C₂₋₁₀ alkenyl; C₂₋₁₀ alkinyl; aryl; aryl-C₁₋₁₀ alkyl; C₃₋₁₀ heterocyclyl; C₅₋₁₀ heteroaryl; halo, CF₃; NO₂; NHC(O)R*, OR, said alkyl, alkenyl, alkinyl, aryl, arylalkyl, heterocyclyl, or heteroaryl being optionally substituted;
R₃, R₅, and R₆ independently represent hydrogen, C₁₋₁₀alkyl; C₂₋₁₀ alkenyl; C₂₋₁₀ alkinyl; aryl; aryl-C₁₋₁₀alkyl; C₃₋₁₀ heterocyclyl; C₅₋₁₀ heteroaryl; halo, CF₃; NO₂; NHC(O)R*, OR, said alkyl, alkenyl, alkinyl, aryl, heterocyclyl, or heteroaryl being optionally substituted; or
R₅ and R₆ together form a 5- or 6-member aryl, heterocyclyl or heteroaryl group;
R is hydrogen or C₁₋₆ alkyl;
R* is hydrogen, or C₁₋₆ alkyl, or OH,
wherein the optional substituents are preferably selected from the group of one to three OH, C₁₋₆ alkyl, halo, NO₂, C₁₋₆ alkoxy, and CF₃,
or a pharmaceutically acceptable salt thereof.

21. The method of claim 20 wherein said cell is contacted by a compound of formula Ia 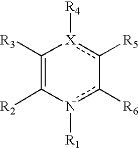
wherein
X is C or N;
------ is an optional double bond;
R₁ and R₄ independently represent hydrogen, a chemical bond, C₁₋₁₀alkyl; C₂₋₁₀ alkenyl; C₂₋₁₀ alkinyl; aryl; aryl-C₁₋₁₀alkyl; C₃₋₁₀ heterocyclyl; C₅₋₁₀ heteroaryl; halo, CF₃; NO₂; NHC(O)R*, OR, said alkyl, alkenyl, alkinyl, aryl, arylalkyl, heterocyclyl, or heteroaryl being optionally substituted;
R₂, R₃, R₅, and R₆ independently represent hydrogen, C₁₋₁₀alkyl; C₂₋₁₀ alkenyl; C₂₋₁₀ alkinyl; aryl; aryl-C₁₋₁₀alkyl; C₃₋₁₀ heterocyclyl; C₅₋₁₀ heteroaryl; halo, CF₃; NO₂; NHC(O)R*, OR, said alkyl, alkenyl, alkinyl, aryl, heterocyclyl, or heteroaryl being optionally substituted; or
R₅ and R₆ together form a 5- or 6-member aryl, heterocyclyl or heteroaryl group;
R is hydrogen or C₁₋₆ alkyl;
R* is hydrogen, or C₁₋₆ alkyl, or OH,
wherein the optional substituents are preferably selected from the group of one to three OH, C₁₋₆ alkyl, halo, NO₂, C₁₋₆ alkoxy, and CF₃,
or a pharmaceutically acceptable salt thereof.

22. The method of claim 20 wherein said cell is contacted with a compound of formula Ib 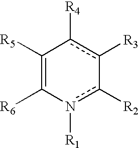
wherein R₁, R₂, R₃, R₄, R₅, and R₆ are as defined in claim 19.

23. The method of claim 20 wherein said cell is contacted with a compound of formula Ic 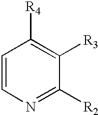
wherein R₂, R₃, and R₄ are as defined in claim 1.

24. A method of inhibiting the vascularization of endothelial cells, comprising contacting an endothelial cell, or a tissue or organ comprising endothelial cells, with an effective amount of a compound of formula I 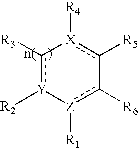
wherein
X, Y and Z independently represent C or N;
------ is an optional double bond;
n is 0 or 1;
R₁, R₂, and R₄ independently represent hydrogen, a chemical bond, C₁₋₁₀ alkyl; C₂₋₁₀ alkenyl; C₂₋₁₀ alkinyl; aryl; aryl-C₁₋₁₀ alkyl; C₃₋₁₀ heterocyclyl; C₅₋₁₀ heteroaryl; halo, CF₃; NO₂; NHC(O)R*, OR, said alkyl, alkenyl, alkinyl, aryl, arylalkyl, heterocyclyl, or heteroaryl being optionally substituted;
R₃, R₅, and R₆ independently represent hydrogen, C₁₋₁₀alkyl; C₂₋₁₀ alkenyl; C₂₋₁₀ alkinyl; aryl; aryl-C₁₋₁₀alkyl; C₃₋₁₀ heterocyclyl; C₅₋₁₀ heteroaryl; halo, CF₃; NO₂; NHC(O)R*, OR, said alkyl, alkenyl, alkinyl, aryl, heterocyclyl, or heteroaryl being optionally substituted; or
R₅ and R₆ together form a 5- or 6-member aryl, heterocyclyl or heteroaryl group;
R is hydrogen or C₁₋₆ alkyl;
R* is hydrogen, or C₁₋₆ alkyl, or OH,
wherein the optional substituents are preferably selected from the group of one to three OH, C₁₋₆ alkyl, halo, NO₂, C₁₋₆ alkoxy, and CF₃,
or a pharmaceutically acceptable salt thereof.

25. The method of claim 24 wherein said cell is contacted with a compound of formula Ia 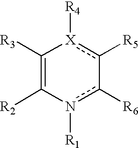
wherein
X is C or N;
------ is an optional double bond;
R₁ and R₄ independently represent hydrogen, a chemical bond, C₁₋₁₀alkyl; C₂₋₁₀ alkenyl; C₂₋₁₀ alkinyl; aryl; aryl-C₁₋₁₀alkyl; C₃₋₁₀ heterocyclyl; C₅₋₁₀ heteroaryl; halo, CF₃; NO₂; NHC(O)R*, OR, said alkyl, alkenyl, alkinyl, aryl, arylalkyl, heterocyclyl, or heteroaryl being optionally substituted;
R₂, R₃, R₅, and R₆ independently represent hydrogen, C₁₋₁₀alkyl; C₂₋₁₀ alkenyl; C₂₋₁₀ alkinyl; aryl; aryl-C₁₋₁₀alkyl; C₃₋₁₀ heterocyclyl; C₅₋₁₀ heteroaryl; halo, CF₃; NO₂; NHC(O)R*, OR, said alkyl, alkenyl, alkinyl, aryl, heterocyclyl, or heteroaryl being optionally substituted; or
R₅ and R₆ together form a 5- or 6-member aryl, heterocyclyl or heteroaryl group;
R is hydrogen or C₁₋₆ alkyl;
R* is hydrogen, or C₁₋₆ alkyl, or OH,
wherein the optional substituents are preferably selected from the group of one to three OH, C₁₋₆ alkyl, halo, NO₂, C₁₋₆ alkoxy, and CF₃,
or a pharmaceutically acceptable salt thereof.

26. A method of treating a disease or condition associated with excessive, unwanted or uncontrolled cell proliferation or angiogenesis in a mammalian subject, comprising administering to the subject an effective amount of a compound of formula I 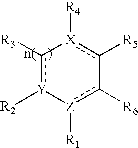
wherein
X, Y and Z independently represent C or N;
------ is an optional double bond;
n is 0 or 1;
R₁, R₂, and R₄ independently represent hydrogen, a chemical bond, C₁₋₁₀ alkyl; C₂₋₁₀ alkenyl; C₂₋₁₀ alkinyl; aryl; aryl-C₁₋₁₀ alkyl; C₃₋₁₀ heterocyclyl; C₅₋₁₀ heteroaryl; halo, CF₃; NO₂; NHC(O)R*, OR, said alkyl, alkenyl, alkinyl, aryl, arylalkyl, heterocyclyl, or heteroaryl being optionally substituted;
R₃, R₅, and R₆ independently represent hydrogen, C₁₋₁₀alkyl; C₂₋₁₀ alkenyl; C₂₋₁₀ alkinyl; aryl; aryl-C₁₋₁₀alkyl; C₃₋₁₀ heterocyclyl; C₅₋₁₀ heteroaryl; halo, CF₃; NO₂; NHC(O)R*, OR, said alkyl, alkenyl, alkinyl, aryl, heterocyclyl, or heteroaryl being optionally substituted; or R₅ and R₆ together form a 5- or 6-member aryl, heterocyclyl or heteroaryl group;
R is hydrogen or C₁₋₆ alkyl;
R* is hydrogen, or C₁₋₆ alkyl, or OH,
wherein the optional substituents are preferably selected from the group of one to three OH, C₁₋₆ alkyl, halo, NO₂, C1-6 alkoxy, and CF₃,
or a pharmaceutically acceptable salt thereof.

27. The method of claim 26 wherein said patient is administered a compound of formula Ia 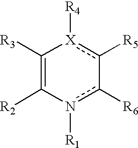
wherein
X is C or N;
------ is an optional double bond;
R₁ and R₄ independently represent hydrogen, a chemical bond, C₁₋₁₀alkyl; C₂₋₁₀ alkenyl; C₂₋₁₀ alkinyl; aryl; aryl-C₁₋₁₀alkyl; C₃₋₁₀ heterocyclyl; C₅₋₁₀ heteroaryl; halo, CF₃; NO₂; NHC(O)R*, OR, said alkyl, alkenyl, alkinyl, aryl, arylalkyl, heterocyclyl, or heteroaryl being optionally substituted;
R₂, R₃, R₅, and R₆ independently represent hydrogen, C₁₋₁₀alkyl; C₂₋₁₀ alkenyl; C₂₋₁₀ alkinyl; aryl; aryl-C₁₋₁₀alkyl; C₃₋₁₀ heterocyclyl; C₅₋₁₀ heteroaryl; halo, CF₃; NO₂; NHC(O)R*, OR, said alkyl, alkenyl, alkinyl, aryl, heterocyclyl, or heteroaryl being optionally substituted; or
R₅ and R₆ together form a 5- or 6-member aryl, heterocyclyl or heteroaryl group;
R is hydrogen or C₁₋₆ alkyl;
R* is hydrogen, or C₁₋₆ alkyl, or OH,
wherein the optional substituents are preferably selected from the group of one to three OH, C₁₋₆ alkyl, halo, NO₂, C₁₋₆ alkoxy, and CF₃,
or a pharmaceutically acceptable salt thereof.

28. The method of claim 27 wherein said disease or condition is selected from the group consisting of malignant tumor growth, diseases associated with corneal neovascularization, arthritis, psoriasis, chronic inflammation, scleroderma, hemangioma, retrolental fibroplasia, abnormal capillary proliferation in hemophiliac joints, and prolonged menstruation and bleeding.

29. The method of claim 28 wherein said disease associated with corneal neovascularization is selected from the group consisting of proliferative retinopathy, retinopathy of prematurity, corneal graft rejection, and neovascular glaucoma.

30. The method of claim 26 wherein said mammalian subject is human.

31. The method of claim 30 wherein said effective amount is in the femtomolar range.

32. The method of claim 30 wherein said effective amount is in the nanomolar range.

33. The method of claim 26 wherein said compound of formula I is administered as a pharmaceutical composition, comprising said compound in admixture with a pharmaceutically acceptable carrier.

34. The method of claim 33 wherein said compound of formula I is administered packaged in a liposome.

35. The method of claim 34 wherein said liposome further comprises an antibody capable of targeted delivery of said compound.

36. The method of claim 26 wherein said disease or condition is cancer, and said compound of formula I is administered intravenously, or by implanting beads impregnated with said compound in said cancer.

37. The method of claim 36 further comprising the administration of a further chemotherapeutic agent for the treatment of said cancer.

38. The method of claim 36 further comprising the administration of a further cytotoxic agent for the treatment of said cancer.

39. A method for prevention of conception comprising administering to a female mammalian subject an effective amount of a compound of formula I 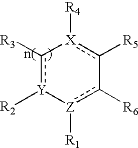
wherein
X, Y and Z independently represent C or N;
------ is an optional double bond;
n is 0 or 1;
R₁, R₂, and R₄ independently represent hydrogen, a chemical bond, C₁₋₁₀ alkyl; C₂₋₁₀ alkenyl; C₂₋₁₀ alkinyl; aryl; aryl-C₁₋₁₀ alkyl; C₃₋₁₀ heterocyclyl; C₅₋₁₀ heteroaryl; halo, CF₃; NO₂; NHC(O)R*, OR, said alkyl, alkenyl, alkinyl, aryl, arylalkyl, heterocyclyl, or heteroaryl being optionally substituted;
R₃, R₅, and R₆ independently represent hydrogen, C₁₋₁₀alkyl; C₂₋₁₀ alkenyl; C₂₋₁₀ alkinyl; aryl; aryl-C₁₋₁₀alkyl; C₃₋₁₀ heterocyclyl; C₅₋₁₀ heteroaryl; halo, CF₃; NO₂; NHC(O)R*, OR, said alkyl, alkenyl, alkinyl, aryl, heterocyclyl, or heteroaryl being optionally substituted; or
R₅ and R₆ together form a 5- or 6-member aryl, heterocyclyl or heteroaryl group;
R is hydrogen or C₁₋₆ alkyl;
R* is hydrogen, or C₁₋₆ alkyl, or OH,
wherein the optional substituents are preferably selected from the group of one to three OH, C₁₋₆ alkyl, halo, NO₂, C₁₋₆ alkoxy, and CF₃,
or a pharmaceutically acceptable salt thereof.

40. The method of claim 39 wherein said subject is administered a compound of formula Ia
wherein
X is C or N;
------ is an optional double bond;
R₁ and R₄ independently represent hydrogen, a chemical bond, C₁₋₁₀alkyl; C₂₋₁₀ alkenyl; C₂₋₁₀ alkinyl; aryl; aryl-C₁₋₁₀alkyl; C₃₋₁₀ heterocyclyl; C₅₋₁₀ heteroaryl; halo, CF₃; NO₂; NHC(O)R*, OR, said alkyl, alkenyl, alkinyl, aryl, arylalkyl, heterocyclyl, or heteroaryl being optionally substituted;
R₂, R₃, R₅, and R₆ independently represent hydrogen, C₁₋₁₀alkyl; C₂₋₁₀ alkenyl; C₂₋₁₀ alkinyl; aryl; aryl-C₁₋₁₀alkyl; C₃₋₁₀ heterocyclyl; C₅₋₁₀ heteroaryl; halo, CF₃; NO₂; NHC(O)R*, OR, said alkyl, alkenyl, alkinyl, aryl, heterocyclyl, or heteroaryl being optionally substituted; or
R₅ and R₆ together form a 5- or 6-member aryl, heterocyclyl or heteroaryl group;
R is hydrogen or C₁₋₆ alkyl;
R* is hydrogen, or C₁₋₆ alkyl, or OH,
wherein the optional substituents are preferably selected from the group of one to three OH, C₁₋₆ alkyl, halo, NO₂, C₁₋₆ alkoxy, and CF₃,
or a pharmaceutically acceptable salt thereof.

41. The method of claim 40 wherein said subject is human.

42. The method of claim 41 wherein said effective amount is in the femtomolar range.

43. The method of claim 41 wherein said effective mount is in the nanomolar range.

44. A method for the inhibition of muscle contraction comprising administering to a subject in need an effective amount of a compound of formula I 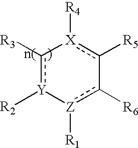
wherein
X, Y and Z independently represent C or N;
------ is an optional double bond;
n is 0 or 1;
R₁, R₂, and R₄ independently represent hydrogen, a chemical bond, C₁₋₁₀ alkyl; C₂₋₁₀ alkenyl; C₂₋₁₀ alkinyl; aryl; aryl-C₁₋₁₀ alkyl; C₃₋₁₀ heterocyclyl; C₅₋₁₀ heteroaryl; halo, CF₃; NO₂; NHC(O)R*, OR, said alkyl, alkenyl, alkinyl, aryl, arylalkyl, heterocyclyl, or heteroaryl being optionally substituted;
R₃, R₅, and R₆ independently represent hydrogen, C₁₋₁₀alkyl; C₂₋₁₀ alkenyl; C₂₋₁₀ alkinyl; aryl; aryl-C₁₋₁₀alkyl; C₃₋₁₀ heterocyclyl; C₅₋₁₀ heteroaryl; halo, CF₃; NO₂; NHC(O)R*, OR, said alkyl, alkenyl, alkinyl, aryl, heterocyclyl, or heteroaryl being optionally substituted; or
R₅ and R₆ together form a 5- or 6-member aryl, heterocyclyl or heteroaryl group;
R is hydrogen or C₁₋₆ alkyl;
R* is hydrogen, or C₁₋₆ alkyl, or OH,
wherein the optional substituents are preferably selected from the group of one to three OH, C₁₋₆ alkyl, halo, NO₂, C₁₋₆ alkoxy, and CF₃,
or a pharmaceutically acceptable salt thereof.

45. The method of claim 44 wherein said subject is administered a compound of formula Ia 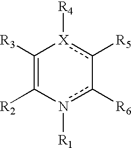
wherein
X is C or N;
------ is an optional double bond;
R₁ and R₄ independently represent hydrogen, a chemical bond, C₁₋₁₀alkyl; C₂₋₁₀ alkenyl; C₂₋₁₀ alkinyl; aryl; aryl-C₁₋₁₀alkyl; C₃₋₁₀ heterocyclyl; C₅₋₁₀ heteroaryl; halo, CF₃; NO₂; NHC(O)R*, OR, said alkyl, alkenyl, alkinyl, aryl, arylalkyl, heterocyclyl, or heteroaryl being optionally substituted;
R₂, R₃, R₅, and R₆ independently represent hydrogen, C₁₋₁₀alkyl; C₂₋₁₀ alkenyl; C₂₋₁₀ alkinyl; aryl; aryl-C₁₋₁₀alkyl; C₃₋₁₀ heterocyclyl; C₅₋₁₀ heteroaryl; halo, CF₃; NO₂; NHC(O)R*, OR, said alkyl, alkenyl, alkinyl, aryl, heterocyclyl, or heteroaryl being optionally substituted; or
R₅ and R₆ together form a 5- or 6-member aryl, heterocyclyl or heteroaryl group;
R is hydrogen or C₁₋₆ alkyl;
R* is hydrogen, or C₁₋₆ alkyl, or OH,
wherein the optional substituents are preferably selected from the group of one to three OH, C₁₋₆ alkyl, halo, NO₂, C₁₋₆ alkoxy, and CF₃,
or a pharmaceutically acceptable salt thereof.

46. The method of claim 45 wherein said subject is human.

47. The method of claim 46 wherein said effective amount is in the femtomolar range.

48. The method of claim 46 wherein said effective amount if in the nanomolar range.

49. An article of manufacture comprising
a container,
a label on the container, or a package insert within the container, and
a compound of formula I 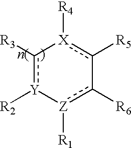
wherein
X, Y and Z independently represent C or N;
------ is an optional double bond;
n is 0 or 1;
R₁, R₂, and R₄ independently represent hydrogen, a chemical bond, C₁₋₁₀ alkyl; C₂₋₁₀ alkenyl; C₂₋₁₀ alkinyl; aryl; aryl-C₁₋₁₀ alkyl; C₃₋₁₀ heterocyclyl; C₅₋₁₀ heteroaryl; halo, CF₃; NO₂; NHC(O)R*, OR, said alkyl, alkenyl, alkinyl, aryl, arylalkyl, heterocyclyl, or heteroaryl being optionally substituted;
R₃, R₅, and R₆ independently represent hydrogen, C₁₋₁₀alkyl; C₂₋₁₀ alkenyl; C₂₋₁₀ alkinyl; aryl; aryl-C₁₋₁₀alkyl; C₃₋₁₀ heterocyclyl; C₅₋₁₀ heteroaryl; halo, CF₃; NO₂; NHC(O)R*, OR, said alkyl, alkenyl, alkinyl, aryl, heterocyclyl, or heteroaryl being optionally substituted; or
R₅ and R₆ together form a 5- or 6-member aryl, heterocyclyl or heteroaryl group;
R is hydrogen or C₁₋₆ alkyl;
R* is hydrogen, or C₁₋₆ alkyl, or OH,
wherein the optional substituents are preferably selected from the group of one to three OH, C₁₋₆ alkyl, halo, NO₂, C₁₋₆ alkoxy, and CF₃,
or a pharmaceutically acceptable salt thereof, within said container,
wherein the composition is effective for inhibiting cell proliferation wherein the label or package insert indicates that the composition is effective for treating a condition characterized by excessive, unwanted or uncontrolled cell growth.

50. The article of manufacture of claim 49 comprising a compound of formula Ia 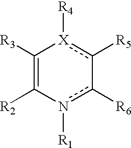
wherein
X is C or N;
------ is an optional double bond;
R₁ and R₄ independently represent hydrogen, a chemical bond, C₁₋₁₀alkyl; C₂₋₁₀ alkenyl; C₂₋₁₀ alkinyl; aryl; aryl-C₁₋₁₀alkyl; C₃₋₁₀ heterocyclyl; C₅₋₁₀ heteroaryl; halo, CF₃; NO₂; NHC(O)R*, OR, said alkyl, alkenyl, alkinyl, aryl, arylalkyl, heterocyclyl, or heteroaryl being optionally substituted;
R₂, R₃, R₅, and R₆ independently represent hydrogen, C₁₋₁₀alkyl; C₂₋₁₀ alkenyl; C₂₋₁₀ alkinyl; aryl; aryl-C₁₋₁₀alkyl; C₃₋₁₀ heterocyclyl; C₅₋₁₀ heteroaryl; halo, CF₃; NO₂; NHC(O)R*, OR, said alkyl, alkenyl, alkinyl, aryl, heterocyclyl, or heteroaryl being optionally substituted; or
R₅ and R₆ together form a 5- or 6-member aryl, heterocyclyl or heteroaryl group;
R is hydrogen or C₁₋₆ alkyl;
R* is hydrogen, or C₁₋₆ alkyl, or OH,
wherein the optional substituents are preferably selected from the group of one to three OH, C₁₋₆ alkyl, halo, NO₂, C₁₋₆ alkoxy, and CF₃,
or a pharmaceutically acceptable salt thereof.

51. An article of manufacture comprising a container,
a label on the container or a package insert within the container,
and a compound of formula I 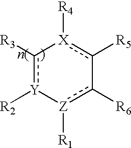
wherein
X, Y and Z independently represent C or N;
------ is an optional double bond;
n is 0 or 1;
R₁, R₂, and R₄ independently represent hydrogen, a chemical bond, C₁₋₁₀ alkyl; C₂₋₁₀ alkenyl; C₂₋₁₀ alkinyl; aryl; aryl-C₁₋₁₀ alkyl; C₃₋₁₀ heterocyclyl; C₅₋₁₀ heteroaryl; halo, CF₃; NO₂; NHC(O)R*, OR, said alkyl, alkenyl, alkinyl, aryl, arylalkyl, heterocyclyl, or heteroaryl being optionally substituted;
R₃, R₅, and R₆ independently represent hydrogen, C₁₋₁₀alkyl; C₂₁ 0 alkenyl;
C₂₋₁₀ alkinyl; aryl; aryl-C₁₋₁₀alkyl; C₃₋₁₀ heterocyclyl; C₅₋₁₀ heteroaryl; halo, CF₃; NO₂; NHC(O)R*, OR, said alkyl, alkenyl, alkinyl, aryl, heterocyclyl, or heteroaryl being optionally substituted; or
R₅ and R₆ together form a 5- or 6-member aryl, heterocyclyl or heteroaryl group;
R is hydrogen or C₁₋₆ alkyl;
R* is hydrogen, or C1-6 alkyl, or OH,
wherein the optional substituents are preferably selected from the group of one to three OH, C₁₋₆ alkyl, halo, NO₂, C₁₋₆ alkoxy, and CF₃,
or a pharmaceutically acceptable salt thereof, within said container,
wherein said label or package insert indicates that the composition is effective for inhibition of angiogenesis.

52. The article of manufacture of claim 51 comprising a compound of formula Ia 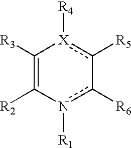
wherein
X is C or N; —
------ is an optional double bond;
R₁ and R₄ independently represent hydrogen, a chemical bond, C₁₋₁₀alkyl; C₂₋₁₀ alkenyl; C₂₋₁₀ alkinyl; aryl; aryl-C₁₋₁₀alkyl; C₃₋₁₀ heterocyclyl; C₅₋₁₀ heteroaryl; halo, CF₃; NO₂; NHC(O)R*, OR, said alkyl, alkenyl, alkinyl, aryl, arylalkyl, heterocyclyl, or heteroaryl being optionally substituted;
R₂, R₃, R₅, and R₆ independently represent hydrogen, C₁₋₁₀alkyl; C₂₋₁₀ alkenyl; C₂₋₁₀ alkinyl; aryl; aryl-C₁₋₁₀alkyl; C₃₋₁₀ heterocyclyl; C₅₋₁₀ heteroaryl; halo, CF₃; NO₂; NHC(O)R*, OR, said alkyl, alkenyl, alkinyl, aryl, heterocyclyl, or heteroaryl being optionally substituted; or
R₅ and R₆ together form a 5- or 6-member aryl, heterocyclyl or heteroaryl group;
R is hydrogen or C₁₋₆ alkyl;
R* is hydrogen, or C₁₋₆ alkyl, or OH,
wherein the optional substituents are preferably selected from the group of one to three OH, C₁₋₆ alkyl, halo, NO₂, C₁₋₆ alkoxy, and CF₃,
or a pharmaceutically acceptable salt thereof.

53. An article of manufacture comprising a container,
a label on the container or a package insert within the container,
and a compound of formula I 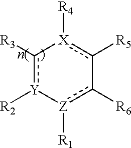
wherein
X, Y and Z independently represent C or N;
------ is an optional double bond;
n is 0 or 1;
R₁, R₂, and R₄ independently represent hydrogen, a chemical bond, C₁₋₁₀ alkyl; C₂₋₁₀ alkenyl; C₂₋₁₀ alkinyl; aryl; aryl-C₁₋₁₀ alkyl; C₃₋₁₀ heterocyclyl; C₅₋₁₀ heteroaryl; halo, CF₃; NO₂; NHC(O)R*, OR, said alkyl, alkenyl, alkinyl, aryl, arylalkyl, heterocyclyl, or heteroaryl being optionally substituted;
R₃, R₅, and R₆ independently represent hydrogen, C₁₋₁₀alkyl; C₂₋₁₀ alkenyl; C₂₋₁₀ alkinyl; aryl; aryl-C₁₋₁₀alkyl; C₃₋₁₀ heterocyclyl; C₅₋₁₀ heteroaryl; halo, CF₃; NO₂; NHC(O)R*, OR, said alkyl, alkenyl, alkinyl, aryl, heterocyclyl, or heteroaryl being optionally substituted; or
R₅ and R₆ together form a 5- or 6-member aryl, heterocyclyl or heteroaryl group;
R is hydrogen or C₁₋₆ alkyl;
R* is hydrogen, or C₁₋₆ alkyl, or OH,
wherein the optional substituents are preferably selected from the group of one to three OH, C₁₋₆ alkyl, halo, NO₂, C₁₋₆ alkoxy, and CF₃,
or a pharmaceutically acceptable salt thereof, within said container, wherein said label or package insert indicates that the composition is effective for the prevention of conception.

54. The article of manufacture of claim 53 comprising a compound of formula Ia 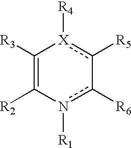
wherein
X is C or N; —
------ is an optional double bond;
R₁ and R₄ independently represent hydrogen, a chemical bond, C₁₋₁₀alkyl; C₂₋₁₀ alkenyl; C₂₋₁₀ alkinyl; aryl; aryl-C₁₋₁₀alkyl; C₃₋₁₀ heterocyclyl; C₅₋₁₀ heteroaryl; halo, CF₃; NO₂; NHC(O)R*, OR, said alkyl, alkenyl, alkinyl, aryl, arylalkyl, heterocyclyl, or heteroaryl being optionally substituted;
R₂, R₃, R₅, and R₆ independently represent hydrogen, C₁₋₁₀alkyl; C₂₋₁₀ alkenyl; C₂₋₁₀ alkinyl; aryl; aryl-C₁₋₁₀alkyl; C₃₋₁₀ heterocyclyl; C₅₋₁₀ heteroaryl; halo, CF₃; NO₂; NHC(O)R*, OR, said alkyl, alkenyl, alkinyl, aryl, heterocyclyl, or heteroaryl being optionally substituted; or
R₅ and R₆ together form a 5- or 6-member aryl, heterocyclyl or heteroaryl group;
R is hydrogen or C₁₋₆ alkyl;
R* is hydrogen, or C₁₋₆ alkyl, or OH,
wherein the optional substituents are preferably selected from the group of one to three OH, C₁₋₆ alkyl, halo, NO₂, C₁₋₆ alkoxy, and CF₃,
or a pharmaceutically acceptable salt thereof.

55. An article of manufacture comprising a container, a label on the container or a package insert within the container, and a compound of formula I 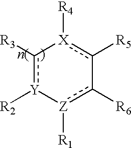
wherein
X, Y and Z independently represent C or N;
------ is an optional double bond;
n is 0 or 1;
R₁, R₂, and R₄ independently represent hydrogen, a chemical bond, C₁₋₁₀ alkyl; C₂₋₁₀ alkenyl; C₂₋₁₀ alkinyl; aryl; aryl-C₁₋₁₀ alkyl; C₃₋₁₀ heterocyclyl; C₅₋₁₀ heteroaryl; halo, CF₃; NO₂; NHC(O)R*, OR, said alkyl, alkenyl, alkinyl, aryl, arylalkyl, heterocyclyl, or heteroaryl being optionally substituted;
R₃, R₅, and R₆ independently represent hydrogen, C₁₋₁₀alkyl; C₂₋₁₀ alkenyl; C₂₋₁₀ alkinyl; aryl; aryl-C₁₋₁₀alkyl; C₃₋₁₀ heterocyclyl; C₅₋₁₀ heteroaryl; halo, CF₃; NO₂; NHC(O)R*, OR, said alkyl, alkenyl, alkinyl, aryl, heterocyclyl, or heteroaryl being optionally substituted; or
R₅ and R₆ together form a 5- or 6-member aryl, heterocyclyl or heteroaryl group;
R is hydrogen or C₁₋₆ alkyl;
R* is hydrogen, or C₁₋₆ alkyl, or OH,
wherein the optional substituents are preferably selected from the group of one to three OH, C₁₋₆ alkyl, halo, NO₂, C₁₋₆ alkoxy, and CF₃,
or a pharmaceutically acceptable salt thereof, within said container,
wherein said label or package insert indicates that the composition is effective for the inhibition of muscle contraction.

56. The article of manufacture of claim 55 comprising a compound of formula Ia 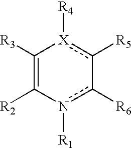
wherein
X is C or N;
------ is an optional double bond;
R₁ and R₄ independently represent hydrogen, a chemical bond, C₁₋₁₀alkyl; C₂₋₁₀ alkenyl; C₂₋₁₀ alkinyl; aryl; aryl-C₁₋₁₀alkyl; C₃₋₁₀ heterocyclyl; C₅₋₁₀ heteroaryl; halo, CF₃; NO₂; NHC(O)R*, OR, said alkyl, alkenyl, alkinyl, aryl, arylalkyl, heterocyclyl, or heteroaryl being optionally substituted;
R₂, R₃, R₅, and R₆ independently represent hydrogen, C₁₋₁₀alkyl; C₂₋₁₀ alkenyl; C₂₋₁₀ alkinyl; aryl; aryl-C₁₋₁₀alkyl; C₃₋₁₀ heterocyclyl; C₅₋₁₀ heteroaryl; halo, CF₃; NO₂; NHC(O)R*, OR, said alkyl, alkenyl, alkinyl, aryl, heterocyclyl, or heteroaryl being optionally substituted; or
R₅ and R₆ together form a 5- or 6-member aryl, heterocyclyl or heteroaryl group;
R is hydrogen or C₁₋₆ alkyl;
R* is hydrogen, or C₁₋₆ alkyl, or OH,
wherein the optional substituents are preferably selected from the group of one to three OH, C₁₋₆ alkyl, halo, NO₂, C₁₋₆ alkoxy, and CF₃,
or a pharmaceutically acceptable salt thereof.
